(19) United States
(12) Patent Application Publication
(10) Pub. No.: US 2022/0265700 A1
(43) Pub. Date: Aug. 25, 2022

(54) METHODS TO PREVENT, INHIBIT OR TREAT INTERVERTEBRAL DISC DEGENERATION

(71) Applicant: University of Iowa Research Foundation, Iowa City, IA (US)

(72) Inventors: Dong Rim Seol, Iowa City, IA (US); James A. Martin, Iowa City, IA (US); Tae-Hong Lim, Coralville, IA (US); Mitchell C. Coleman, Iowa City, IA (US); Aliasger K. Salem, Coralville, IA (US); Ino Song, Iowa City, IA (US)

(21) Appl. No.: 17/580,129

(22) Filed: Jan. 20, 2022

Related U.S. Application Data
(60) Provisional application No. 63/139,437, filed on Jan. 20, 2021.

(51) Int. Cl. A61K 31/728; A61K 9/00; A61K 9/06; A61K 31/515; A61K 47/34; A61K 31/7076; A61K 31/155; A61P 19/02

(52) U.S. Cl. CPC A61K 31/728; A61K 9/0019; A61K 9/06; A61P 19/02; A61K 47/34; A61K 31/7076; A61K 31/155; A61K 31/515

(57) ABSTRACT

A method to prevent, inhibit or treat intervertebral disc disease in a mammal and compositions useful in that regard are provided.

### CROSS-REFERENCE TO RELATED APPLICATIONS

This application claims the benefit of the filing date of U.S. application No. 63/139,437, filed on Jan. 20, 2021, the disclosure of which is incorporated by reference herein.

### STATEMENT OF GOVERNMENT RIGHTS

This invention was made with government support under grant W81XWH2010152 awarded by the Department of Defense. The government has certain rights in the invention.

### BACKGROUND

Intervertebral disc degeneration is clinically related to chronic low back pain, the most prevalent of all musculoskeletal disorders. As such, disc degeneration constitutes a heavy financial and health burden to society (Hong et al., 2013; Katz et al., 2006; Urban et al., 2003). Intervertebral disc degeneration is a complex disorder attributable to many different factors including aging (Miller et al., 1988), abnormal mechanical loading (Adams et al., 2000), limited nutrient supply (Maroudas et al., 1975), trauma (Dudli et al., 2014), and genetic factors (Batie et al., 1995; Sambrook et al., 1999). In particular, traumatic injuries on the spinal joints including disc herniation, which has up to 2% incidence in people aged 30-50 years, can lead to IDD in the adolescent and young adult discs.

There are two ways of treatments, non-surgical treatment and surgical treatment. Surgery is considered when symptoms interfere with activities of daily living and non-surgical treatment has failed. Surgical treatment may be in the form of microdiscectomy, fusion, or artificial disc replacement for treating discogenic pain that attributes the degenerated disc. Minimally invasive therapies for disc herniation such as endoscopic discectomy, percutaneous discectomy, and chemonucleolysis are available as well. The non-surgical treatment, e.g. conservative approach, includes traction, bed rest, heat and ice to the affected area, exercises, and physical therapy. Some patients have anti-inflammatory and muscle relaxant medications, and epidural steroid injections. Unfortunately, there are no effective non-surgical therapies at early stage of discal injury. Thus, development of minimal invasive therapies for acute discal injuries is urgent for preventing disc degeneration progression.

The nucleus pulposus of the intervertebral disc resides in a relatively hypoxic environment due to its avascularity. As a result, notochordal and chondrocyte-like cells primarily utilize glycolysis to produce energy. Oxidative stress has been defined as a result of an imbalance between intracellular oxidants and antioxidants. The roles of oxidative stress have been extensively investigated in different diseases including neurodegenerative diseases (Elfway et al., 2018), cardiovascular diseases (Sahoo et al., 2016; Sena et al., 2018), diabetes (Victor et al., 2011), atherosclerosis (Kattoor et al., 2017) and osteoarthritis (Lepetsos et al., 2016).

### SUMMARY

The disclosure provides for compositions and methods to prevent, inhibit, e.g., delay, or treat the pathogenesis of spinal disorders, e.g., intervertebral disc degeneration. The compositions and methods employ targeting oxidative stress that may slow or prevent disease progression.

As described hereinbelow, the inhibitory effects of amobarbital (Amo) on the mitochondria of nucleus pulposus cells under tert-butyl hydrogen peroxide (tBHP)-induced oxidative stress or in nucleus pulposus tissues under oxidative stress from tissue injury, can be therapeutic targets for disc degeneration. Specifically, the effect of amobarbital as a complex I inhibitor inhibits mitochondria dysfunction in nucleus pulposus cells subjected to traumatic discal injury. In addition, an ex vivo organ culture of rabbit spinal motion segments was tested. Degenerative discs were created by a needle puncture, and cellular and matrix changes after hydrogel injection with or without Amo were evaluated by histological analyses. The efficiency of amobarbital, e.g., to prevent progression of disc degeneration, can be enhanced in a drug delivery system which is locally delivered into target disc tissue, gelled at body temperature, and allows sustained release of drug. For example, a composite hydrogel may be employed which can be injected into the targeted nucleus pulposus using a small size needle to minimize permanent damage in the annulus fibrosus. For example, the hydrogel may include hyaluronic acid (HA) and optionally generic Pluronic® F-127 (poly(ethylene oxide)-poly(propylene oxide)-poly(ethylene oxide) tri-copolymer), allowing for gelling at body temperature. This system allows minimal back-flow of drug after injection and sustained release during polymer degradation.

The present disclosure thus provides an injectable hydrogel composition comprising a polysaccharide, e.g., a natural polysaccharide such as hyaluronic acid, hydroxypropylcellulose, karya gum (KG), guar gum (GUG), or gellan gum (GEG), a semi-synthetic polysaccharide or a synthetic polysaccharide, and a compound useful to prevent, inhibit or treat spinal degeneration, such as an antioxidant or anti-reactive oxygen species (ROS) agent, and optionally a synthetic polymer, e.g., poloxamers (or Pluronics®) such as P407 (F127), P338 (F108), P237 (F87), or P188 (F68), poly(ethylene oxide), poly(N-isopropylacrylamide), poly[2-(dimethylamino)ethyl methacrylate] (pDMAEMA), poly(vinvlcaprolactame), poly(2-isopropyl-2-oxazoline), or poly(vinylmethylether) whose thermoresponsive properties cause the composition to become semi-solid gel once injected into human body (37° C.) and can prevent leakage from the site of injection such as a disc. In one embodiment, the compound reversibly inhibits the respiratory enzyme complex I. In one embodiment, the hydrogel comprises an effective amount of amobarbital, e.g., from about 0.1 mM to about 50 mM or about 0.25 mM to about 10 mM, metformin (N,N,-dimethylbiguanide) a biguanide derivative, N,N-diethylbiguanide, N,N,-dipropylbiguanide, phenformin (Sogame et al., Biopharm. Drug Dispos., 0:476 (2009)), or HL010183 (Koh et al., Bioorg. Med. Chem., 21:2305 (2013)), or adenosine diphosphate ribose or a derivative thereof. In one embodiment, the volume administered is about 0.1 mL to about 15 mL, e.g., about 1 mL to about 10 mL or about 2 mL to about 5 mL. The combination of materials in the hydrogel offers a practical advantage, for instance, in enabling health care providers to protect discs acutely after injury or to inhibit disc degeneration, e.g., degeneration without a known underlying injury.

In one embodiment, the disclosure provides for the use of an injectable hydrogel composition comprising a biopolymer, such as a polysaccharide, a synthetic polymer, and a compound in an amount that optionally reversibly inhibits respiratory enzyme complex I. In one embodiment, the hydrogel includes about 0.2 wt/vol to about 4% wt/vol hyaluronic acid. In one embodiment, the polysaccharide comprises hyaluronic acid. In one embodiment, the synthetic polymer comprises a poloxamer 407 (Pluronics® F127). In one embodiment, the hydrogel includes about 15% wt/vol to about 20% wt/vol poloxamer 407 (Pluronics® F127). In one embodiment, the compound comprises amobarbital. In one embodiment, the hydrogel comprises N-isopropyl acrylamide polymer, ethylhydroxyethylcellulose, poly(etheylene oxide-b-propylene oxide-b-ethylene oxide), poloxamers, Pluronics® polymers, poly(ethylene glycol)/poly(D,L-lactic acid-co-glycolic acid) block copolymers, polysaccharides, alginate, polyphosphazines, polyacrylates, Tetronics™ polymers, or polyethylene oxide-polypropylene glycol block copolymers. In one embodiment, the polysaccharide comprises hyaluronic acid of about or greater than 1.5 M (mega) Dalton (Da). In one embodiment, the molecular weight is about 1,600,000 to 3,900,000, or about 1,900,000 to 3,200,000. In one embodiment, the polysaccharide comprises hydroxypropylcellulose, karya gum (KG), guar gum (GUG), or gellan gum (GEG). In one embodiment, the polysaccharide is present in the hydrogel at about 0.2% (wt/vol) to about 1.0% (wt/vol). In one embodiment, the composition is a reverse temperature-sensitive hydrogel (one that is non-viscous at “low” temperature, e.g., at or below room temperature, e.g., about 70° F. or less. The low initial viscosity allows the hydrogel to coat tissues before it sets (i.e., the viscosity increases at temperatures above room temperature, e.g., about 80° F. or greater including human body temperature such as about 98° F.), which provides for superior retention in and substantially improves the bioavailability of the therapeutic compound dissolved in the gel. Reverse temperature-sensitive hydrogels, which have initial viscosities of about 100 to about 160 or about 80 to about 200, e.g., about 120 to about 140, Pascal Seconds, may be administered using a 22 to 24 gauge needle, e.g., a 22 gauge needle. In contrast, non-reverse temperature-sensitive hydrogels require large bore needles and do not evenly distribute in the joint due to their high initial viscosity.

Also provided is a method to prevent, inhibit or treat spinal disease or degeneration, e.g., intervertebral disc degeneration, due to disease or after injury in a mammal. The method includes administering an effective amount of the composition to a mammal in need thereof. Further provided is a method to inhibit or treat disc degeneration in a mammal. The method includes administering an effective amount of the composition to a mammal having or at risk of having disc degeneration. In one embodiment, the composition comprises hyaluronic acid. In one embodiment, the composition comprises poloxamer 407 (Pluronics® F127). In one embodiment the composition is injected into the mammal. In one embodiment the composition is injected into an intervertebral disc, e.g., nucleus pulposus (inner area of the disc), of a mammal. In one embodiment, the composition comprises amobarbital. In one embodiment, the administration is within 1, 2, 3, 4 or 5 days of an injury. In one embodiment, the administration is with 1, 2, 3, 4, 5, 6, 7, 8, 9, 10 11, or 12 hours of an injury. In one embodiment, a single injection is therapeutically effective.

### DETAILED DESCRIPTION OF THE FIGURES

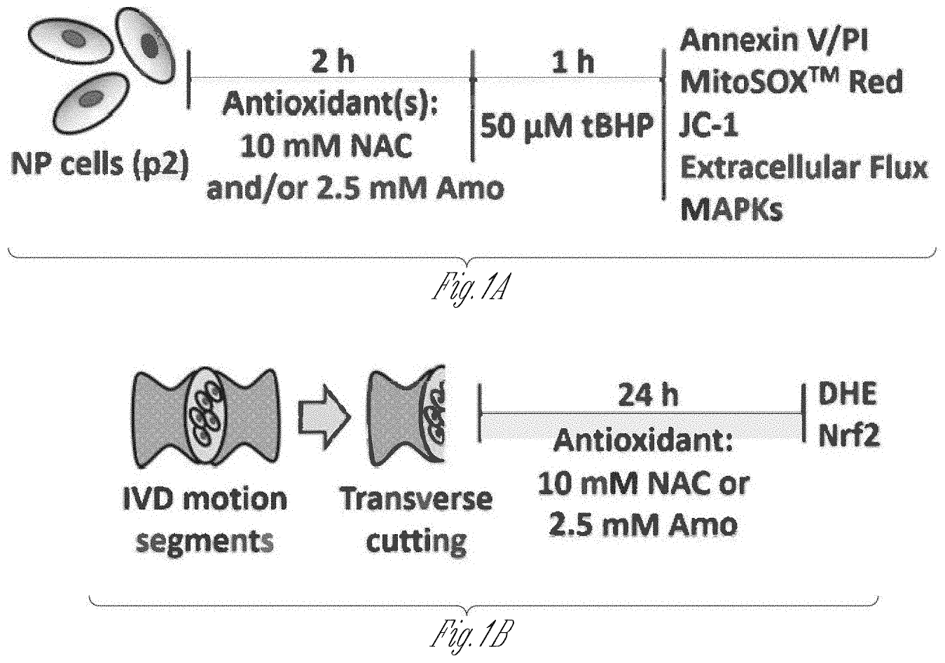

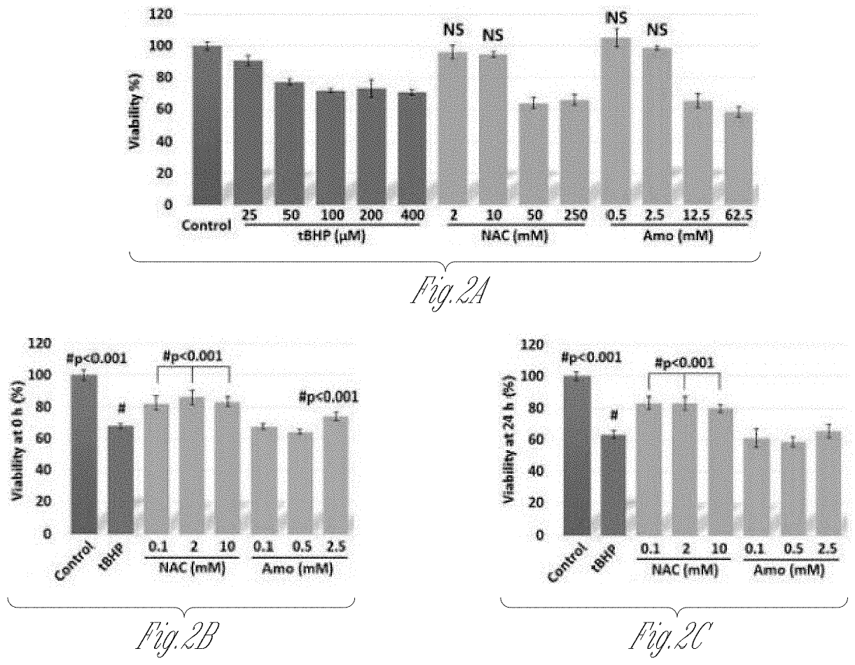

FIGS. 1A-1B. Experimental designs of in vitro (A) and ex vivo (B) studies. NP: nucleus pulposus, NAC: N-acetylcysteine, Amo: amobarbital, tBHP: tert-butyl hydrogen peroxide, IVD: intervertebral disc. MAPK: mitogen-activated protein kinase, DHE: dihydroethidium, Nrf2: nuclear factor (erythroid-derived 2)-like 2.

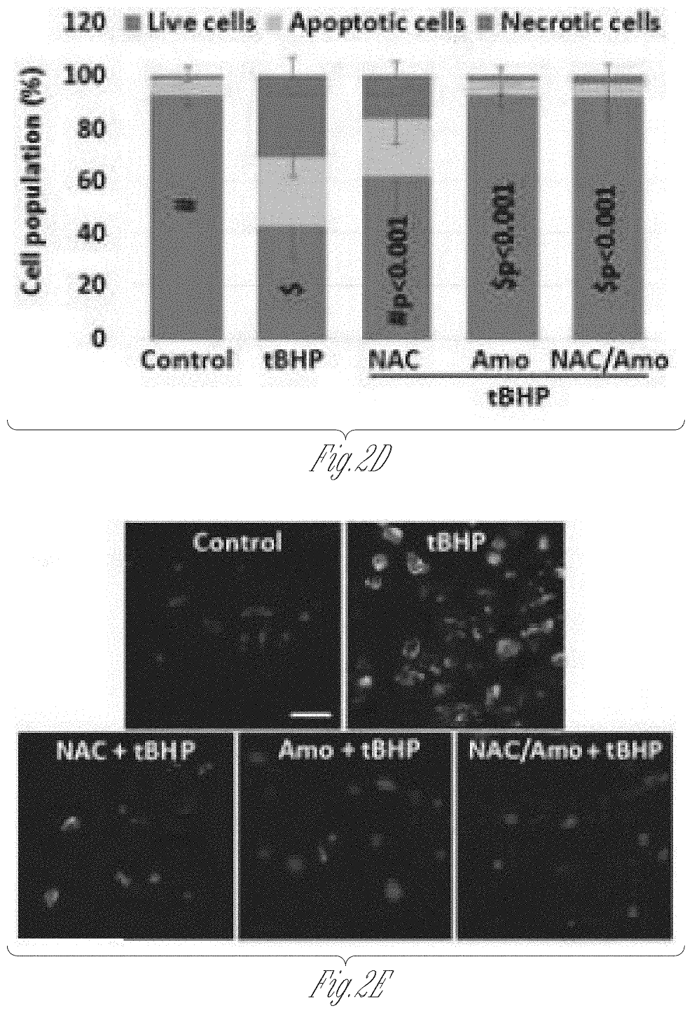

FIGS. 2A-2E. Cytotoxicity of tert-butyl hydrogen peroxide (tBHP), N-acetylcysteine (NAC), and amobarbital (Amo) in nucleus pulposus (NP) cells. (A) Cytotoxicity at various concentrations of tBHP (1 h), NAC (2 h), or Amo (2 h) (n=4, NS: not significant vs. control). (B) Viability of NAC or Amo pre-treatment at 0 h (n=4). (C) Cytotoxicity of NAC or Amo pre-treatment at 24 h (n=4). (D) Quantified population of apoptotic and necrotic cells via Annexin V/propidium iodine (PI) staining. Cells were pre-treated with 10 mM NAC and/or 2.5 mM Amo prior to 50 μM tBHP exposure for 1 h (n=4). (E) Representative images of Annexin V/PI (scale bar=50 μm, blue: DAPI, green: Annexin V, red: PI).

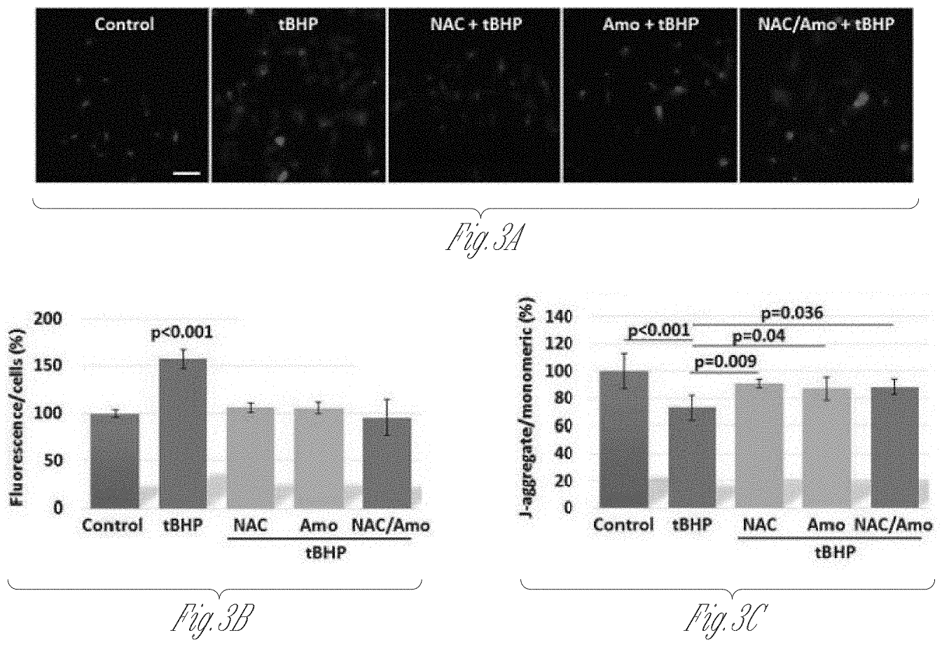

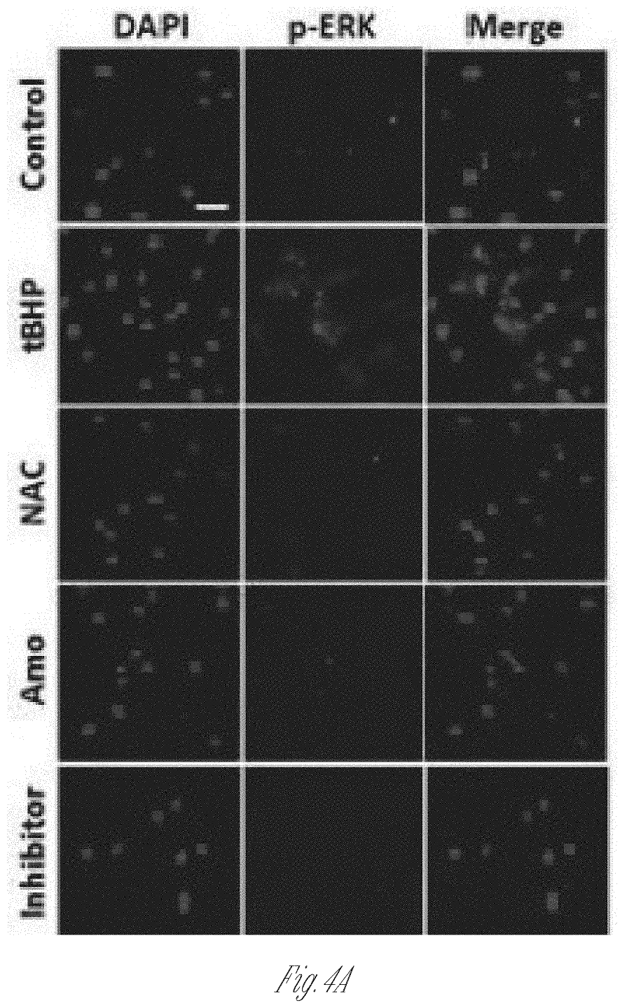

FIGS. 3A-3C. Effect of amobarbital (Amo) in nucleus pulposus (NP) cells. NP cells were pre-treated with 10 mM N-acetylcysteine (NAC) and/or 2.5 mM Amo for 2 h prior to 50 μM tert-butyl hydrogen peroxide (tBHP) exposure for 1 h. (A) Mitochondrial reactive oxygen species (ROS) production via MitoSOX Red staining (scale bar=100 μm, blue: DAPI, red: MitoSOX Red). (B) Quantified MitoSOX Red by a fluorescence plate reader (n=4). (C) Mitochondrial membrane potential via JC-1 staining (n=6).

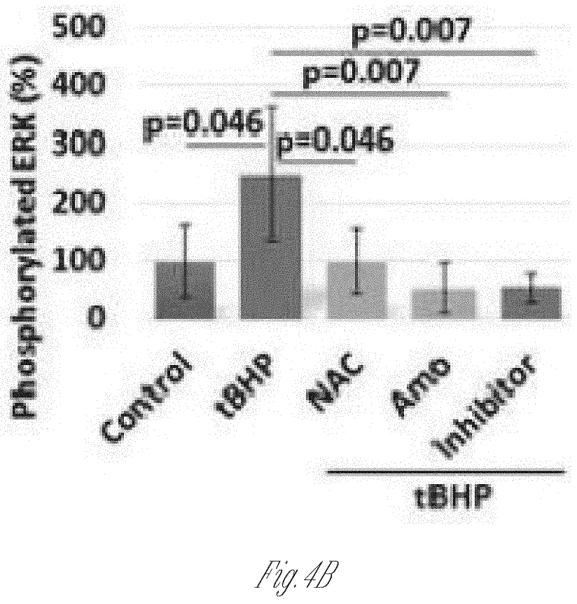

FIGS. 4A-4F. Mitogen-activated protein kinase (MAPK) signaling pathway. Nucleus pulposus cells were pre-treated with 10 mM N-acetylcysteine (NAC) and/or 2.5 mM amobarbital (Amo) for 2 h prior to 50 μM tert-butyl hydrogen peroxide (tBHP) exposure for 1 h. (A) Immunofluorescence (IF) staining of phosphorylated extracellular signal-regulated kinase (p-ERK) (scale bar=50 μm, inhibitor: 20 μM PD98059, blue: DAPI, green: p-ERK). (B) Quantified rate of p-ERK/DAPI (n=4). (C) IF staining of phosphorylated c-JUN N-terminal kinase (QNK) (scale bar=50 μm, inhibitor: 20 μM SB203580, blue: DAPI, green: p-JNK). (D) Quantified rate of p-JNK/DAPI (n=4-5). (E) IF staining of phosphorylated p38 (scale bar=50 μm, inhibitor: 20 μM SP600125, blue: DAPI, green: p-p38). (f) Quantified rate of p-p38/DAPI (n=4).

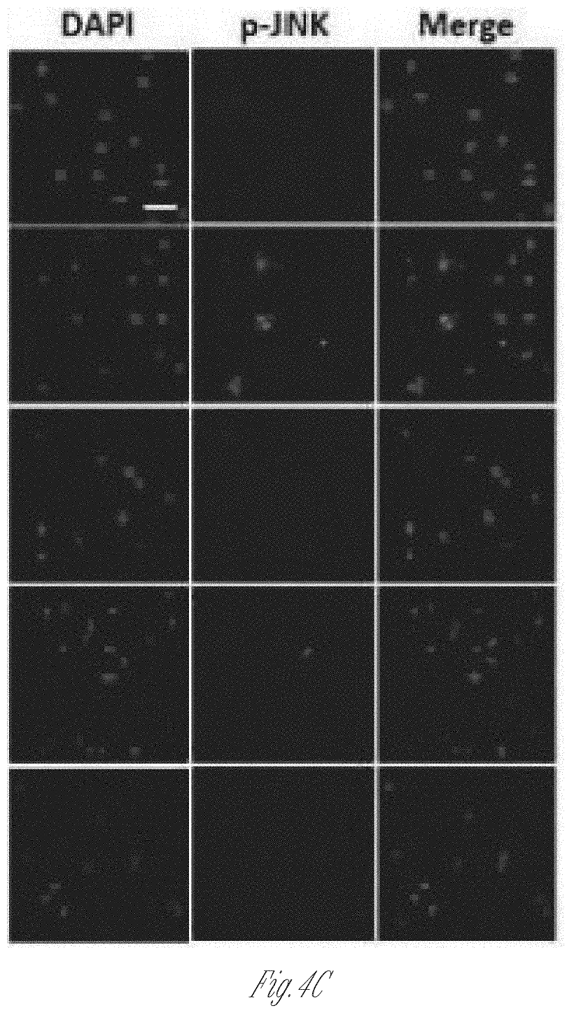

FIGS. 5A-5D. Effect of amobarbital (Amo) in nucleus pulposus (NP) tissues. Oxidative stress was induced by traumatically transverse cutting NP tissues and 10 mM N-acetylcysteine (NAC) or 2.5 mM Amo was given after cutting, for 24 h. (A) Dihydroethidium (DHE) staining (scale bar=50 μm, green: Calcein AM, red: DHE, orange, merged). (B) Quantified rate of DHE/Calcein AM (n=4). (C) Nuclear factor (erythroid-derived 2)-like 2 (Nrf2) staining (scale bar=50 μm, green: Nrf2, blue: DAPI). (D) Quantified rate of number of nuclear and cytosolic Nrf2/DAPI (n=4-6).

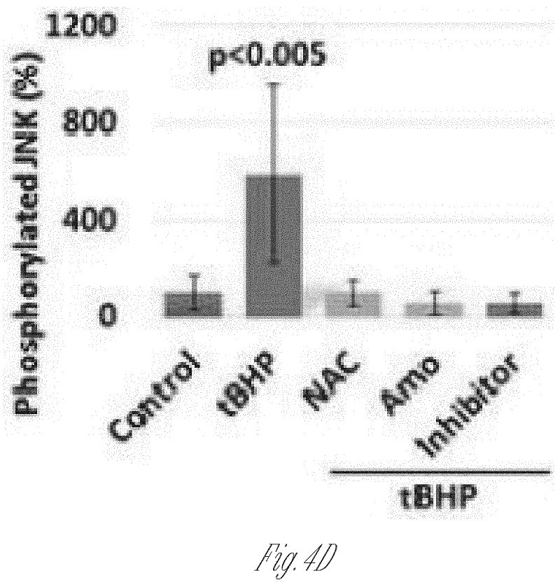

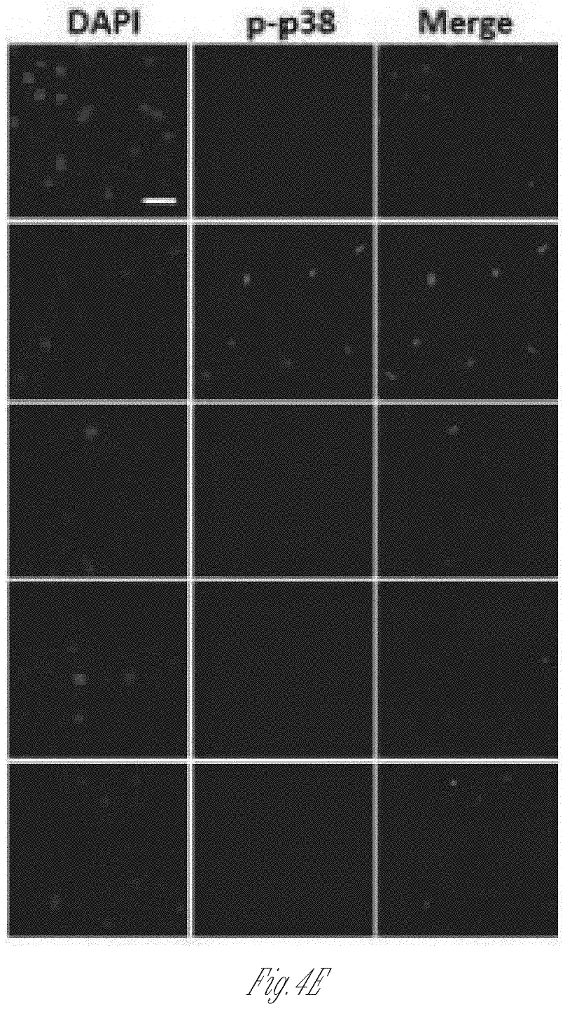

FIGS. 6A-6B. Determination of tert-butyl hydrogen peroxide (tBHP) concentration. (A) Annexin V (green)/propidium iodine (PI: red)/DAPI (blue) staining after 100 μM tBHP exposure (scale bar=100 μm). (B) Quantified mitochondrial reactive oxygen species (ROS) production via MitoSOX Red staining after 25 or 50 μM tBHP exposure (n=4).

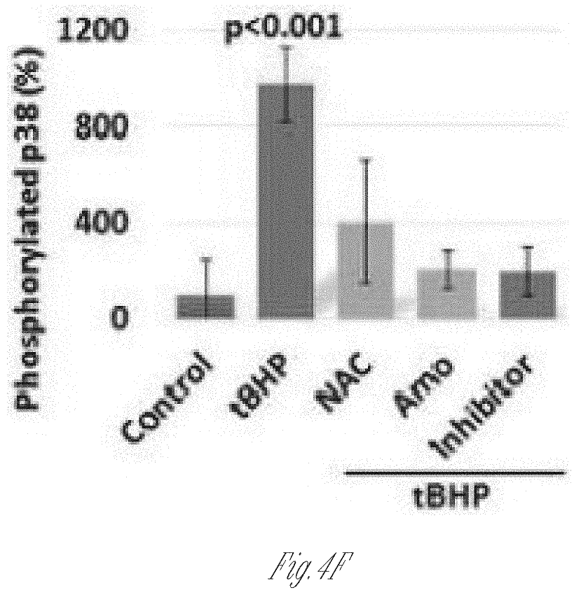

FIGS. 7A-7D. Post-treatment effect of amobarbital (Amo) in tert-butyl hydrogen peroxide (tBHP)-exposed NP cells. The cells were treated with 10 mM N-acetylcysteine (NAC) or 2.5 mM Amo for 2 h after 50 μM tBHP exposure for 1 h. (A) Cytotoxicity (n=4). (B) Cell population of live, apoptosis, and necrosis by Annexin V/propidium iodine (PI)/DAPI staining (n=4). (C) Mitochondrial reactive oxygen species (ROS) production via MitoSOX Red staining (n=4). (D) Mitochondrial membrane potential via JC-1 staining (n=6).

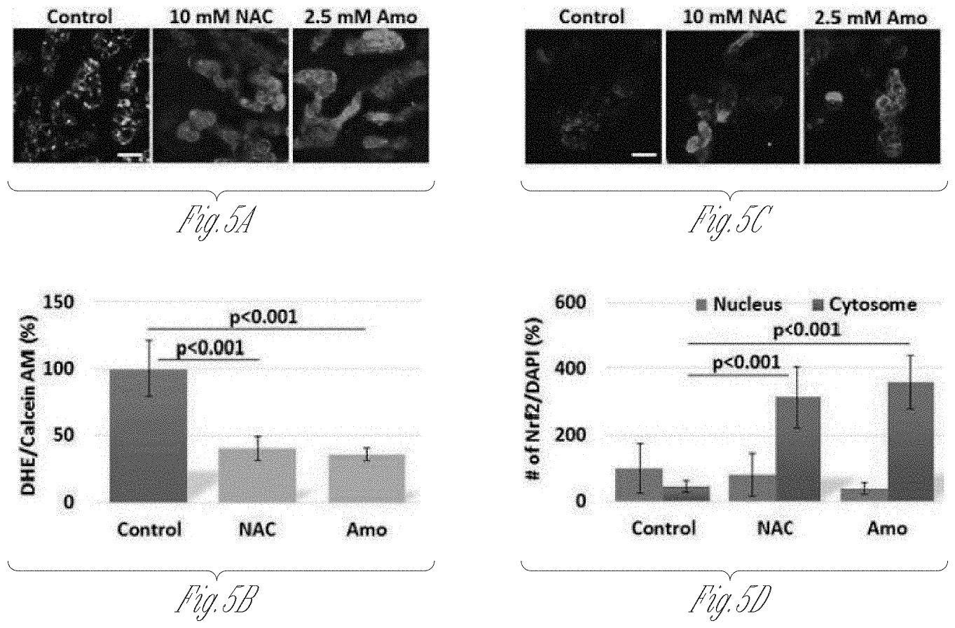

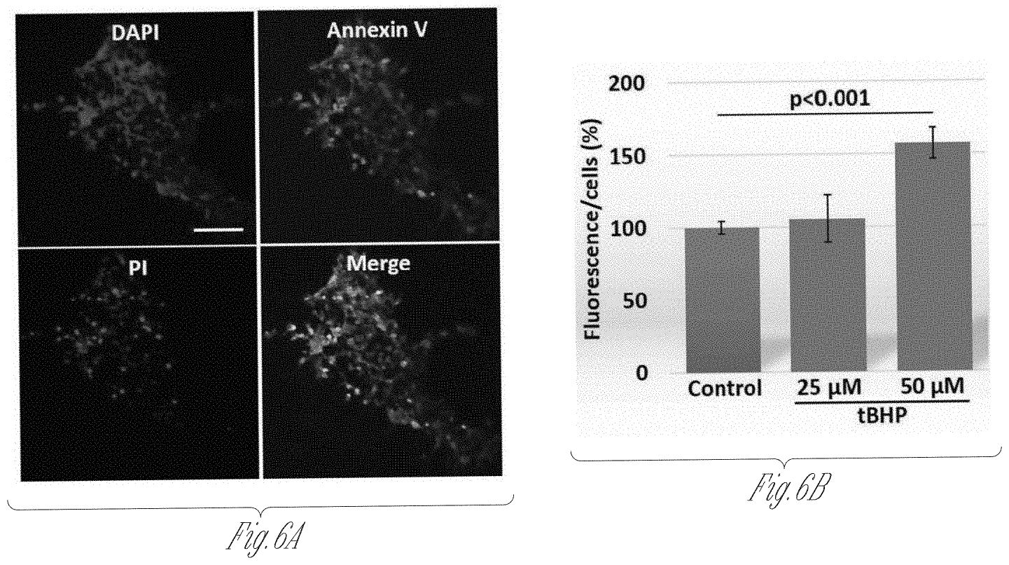

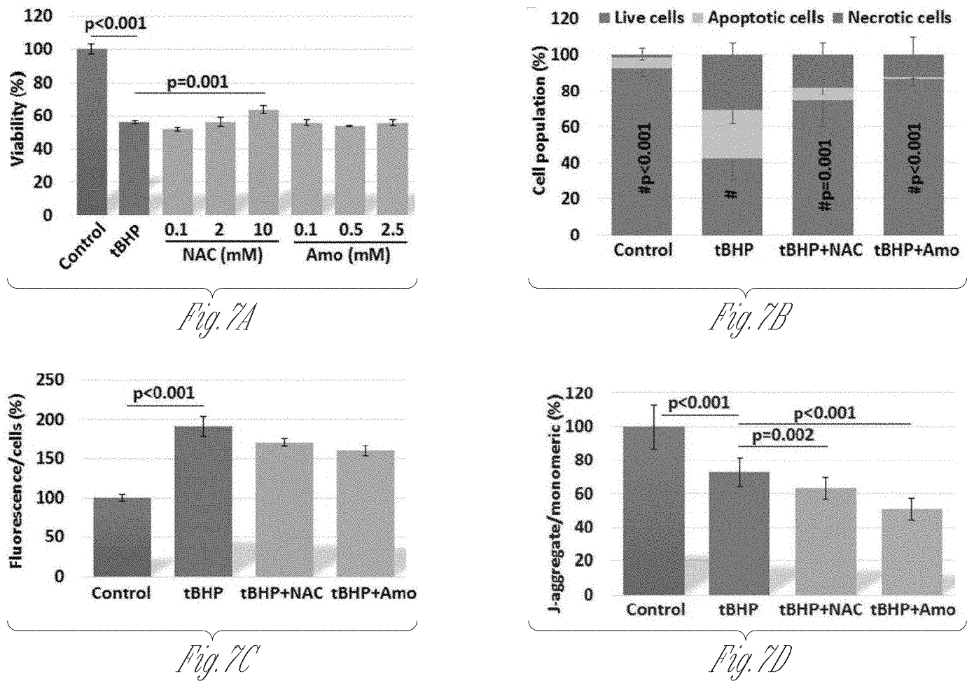

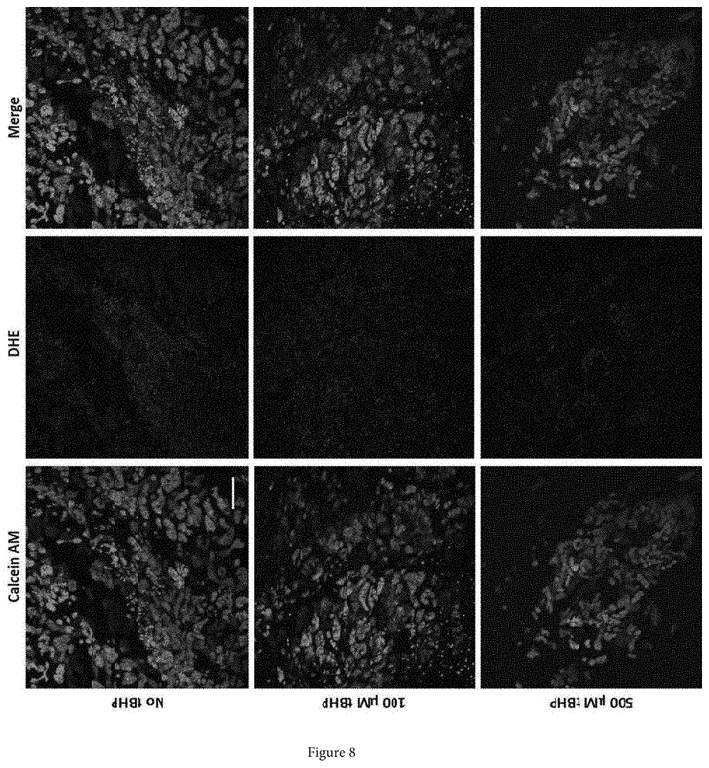

FIG. 8. Dihydroethidium (DHE) staining in nucleus pulposus (NP) tissues after tert-butyl hydrogen peroxide (tBHP) exposure. Oxidative stress was induced by traumatically transverse cutting and/or tBHP (100 or 500 μM for 1 h), and the tissues were stained at 24 h post-injury (scale bar=50 μm, green: Calcein AM, red: DHE).

FIG. 9. Nuclear factor erythroid 2-related factor 2 (Nrf2) signaling pathway under tert-butyl hydrogen peroxide (tBHP)-induced oxidative stress. NP cells were pre-treated with 10 mM N-acetylcysteine (NAC) or 2.5 mM amobarbital (Amo) for 2 h prior to 50 μM tBHP exposure for 1 h (scale bar=100 μm, blue: DAPI, green: Nrf2, n=3-4).

FIGS. 10A-10B. N-acetylcysteine (NAC) treatment decreases dihydroethidium (DHE) staining of IVDs. (A) 100 μM tert-butyl hydrogen peroxide (tBHP) treatment causes significant increase of DHE staining (red). (B) 100 μM tBHP+10 mM NAC shows less oxidation of DHE. Green is live cell staining via Calcein AM. Scale bar=50 μm.

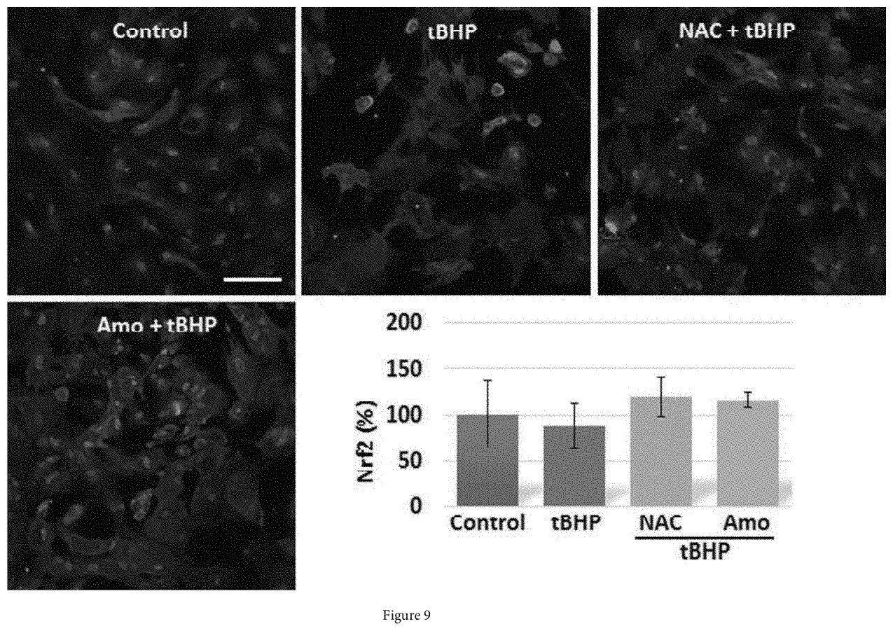

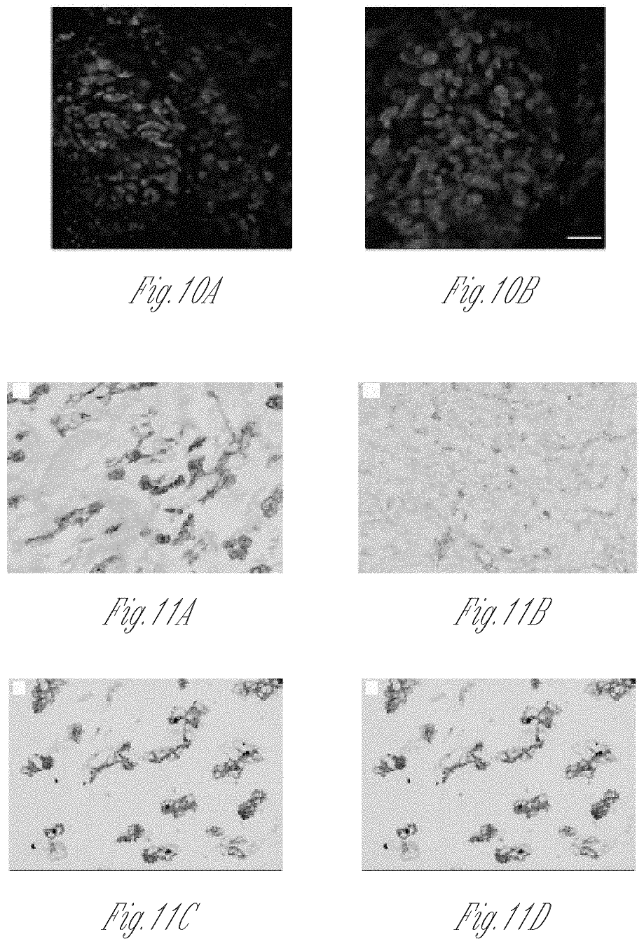

FIGS. 11A-11D. N-acetylcysteine (NAC) treatment decreases superoxide dismutase 2 (SOD2) (A and B) and nuclear factor erythroid 2-related factor 2 (Nrf2) (C and D) immunohistochemistry (IHC) staining after 100 μM tert-butyl hydrogen peroxide (tBHP). Panels A and C show 100 μM tBHP treated and panels B and D show 100 μM tBHP+10 mM NAC treatment. Scale bar=50 μm.

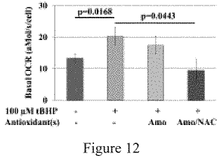

FIG. 12. Basal oxygen consumption rate (OCR) is increased in rabbit NP cells exposed to 100 μM tert-butyl hydrogen peroxide (tBHP). This increase is mitigated with combination treatment with 2.5 mM amobarbital (Amo) and 10 mM N-acetylcysteine (NAC) but not treatment with amobarbital alone. N=3-6.

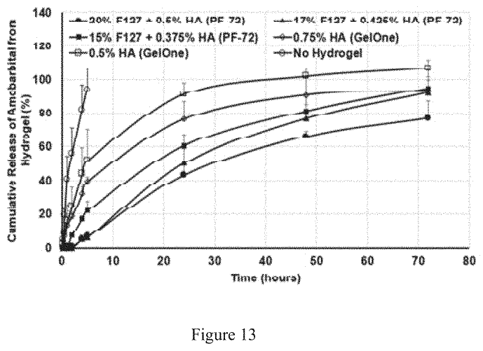

FIG. 13. Cumulative release of amobarbital from hydrogel. The hydrogel was used from PF-72 and Gel-One® with various concentration of F127 and/or hyaluronic acid (HA). Amobarbital without hydrogel was used for a control. All hydrogel system allowed sustained release of amobarbital for 72 hours. N=3.

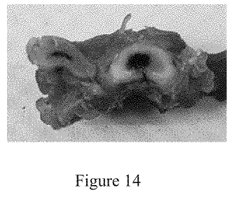

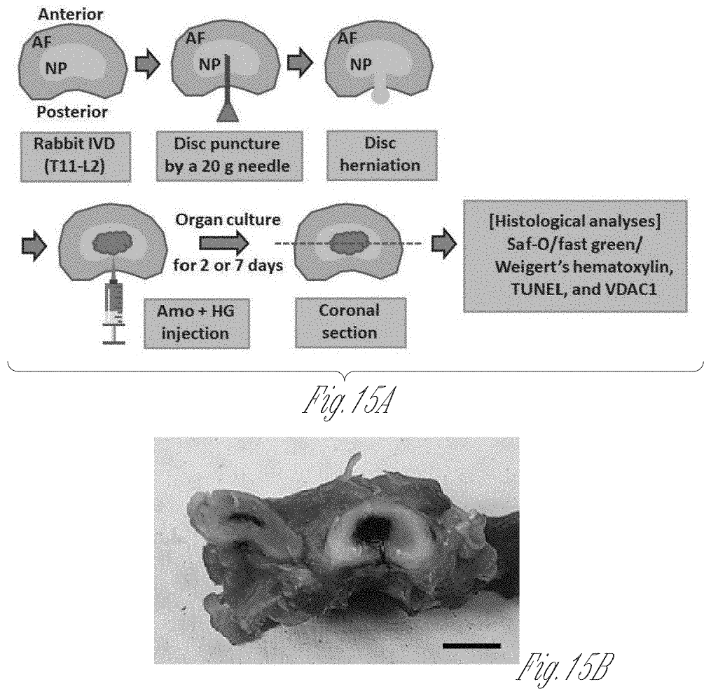

FIG. 14. Feasibility of amobarbital-loaded hydrogel injection into the rabbit nucleus pulposus. Richardson staining was used to visualize the efficiency of local delivery instead of amobarbital and loaded in 17% (wt/vol) F-127/0.425% (wt/vol) hyaluronic acid hydrogel. The hydrogel was locally delivered into the nucleus pulposus and uniformly distributed with minimal back flow.

FIGS. 15A-15B. An experimental design of ex vivo organ culture of rabbit spine motion segments. (A) Ex vivo disc puncture model. AF: annulus fibrosus, NP: nucleus pulposus, IVD: intervertebral disc, T11: eleventh thoracic spine, L2: second lumbar spine, 20 g: 20 gauge, Amo: amobarbital, HG: hydrogel, Saf-O: Safranin-O, TUNEL: terminal deoxynucleotidyl transferase dUTP nick end labelling, VDAC1: voltage-dependent anion channel 1. (B) Validation of HG injection into the rabbit NP. Richardson's dye (blue) was mixed in HG. Scale bar=5 mm.

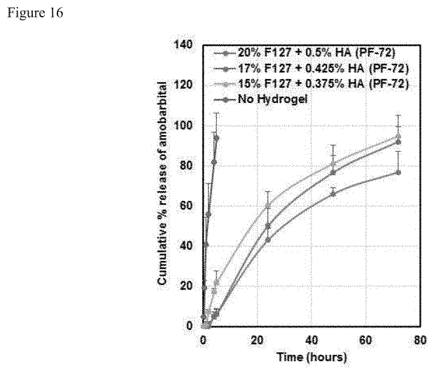

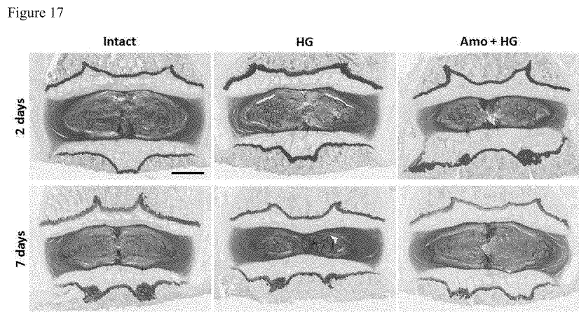

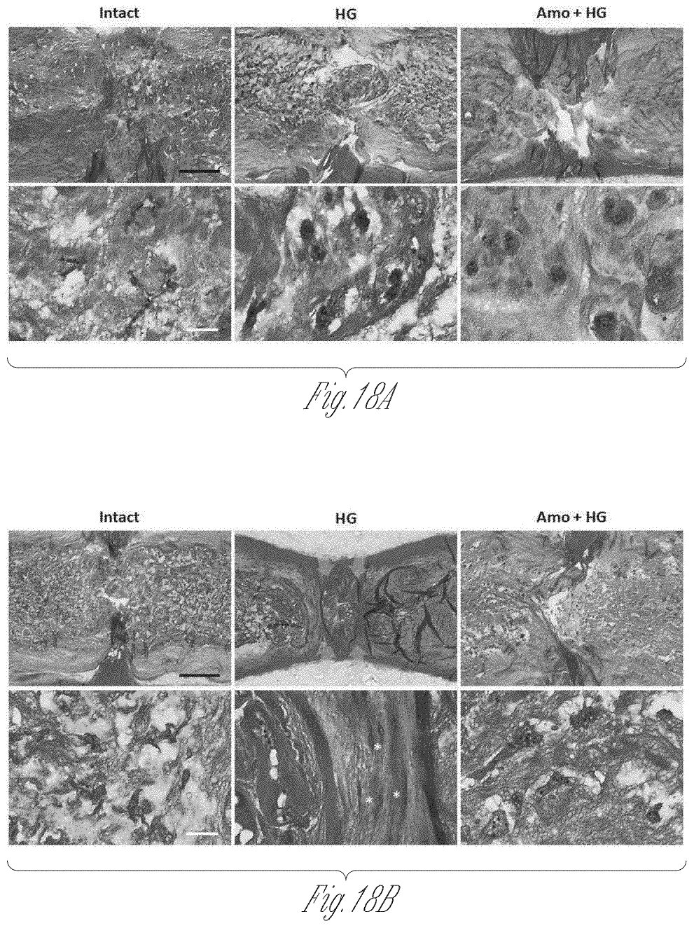

FIG. 16. In vitro amobarbital (Amo) release profile. Amo (2.5 mM) solution was added in lyophilized PF-72® (mixture of F-127 and hyaluronic acid (HA)) and drug release was evaluated by a dialysis membrane diffusion method (n=3).

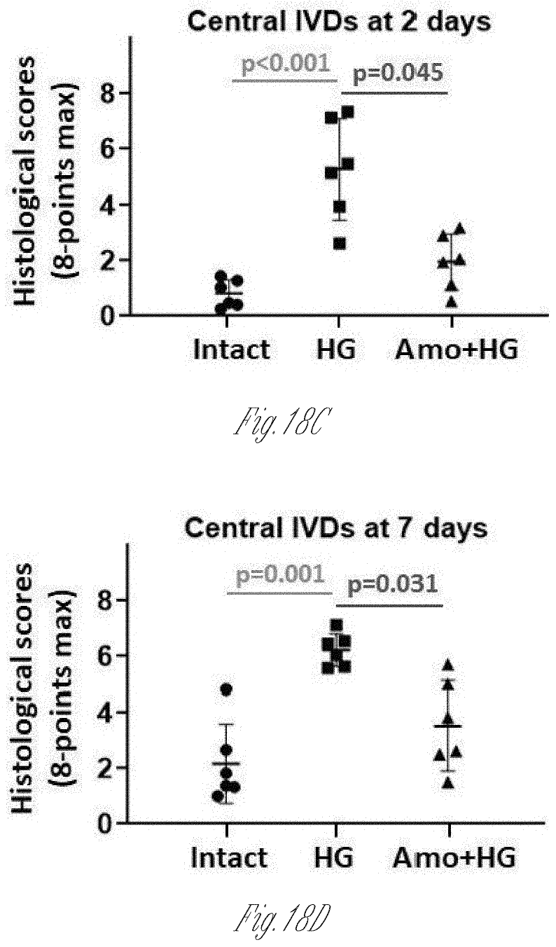

FIG. 17. Whole histological images of intervertebral disc (IVD) degeneration. Disc degeneration was induced in rabbit IVDs using a needle puncture in the central IVD and harvested at 2 and 7 days. The discs were stained with Safranin-O (red), Fast Green (light blue), and Weigert's hematoxylin (blue-black). Intact: no needle puncture with no injection, HG: hydrogel, Amo+HG: amobarbital in hydrogel. Scale bar=2 mm.

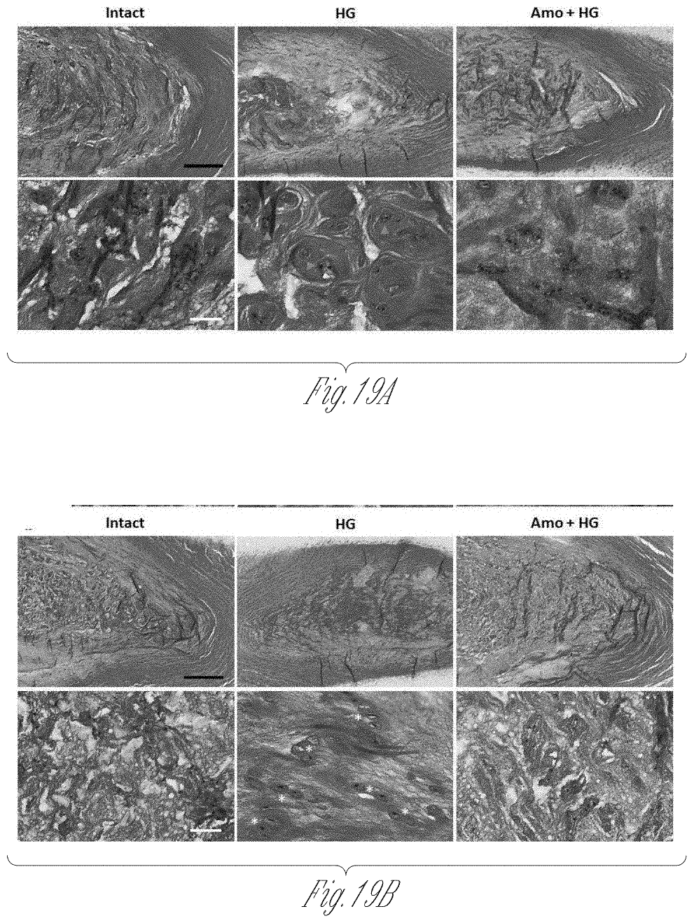

FIGS. 18A-18D. Histological examination of disc degeneration in the central intervertebral discs (IVDs). Extracellular matrix (ECM: upper panel) and cellular (lower panel) changes at (A) 2 and (B) 7 days. Blue arrowheads: clustered cells, yellow arrow bars: fibrous ECM, white asterisks: migrated endplate chondrocytes. (C) Histologic grading at 2 days (n=6). (D) Histologic grading at 6 days (n=6). Intact: no needle puncture with no injection, HG: a needle puncture with hydrogel injection, Amo+HG: a needle puncture with amobarbital+hydrogel injection. Black scale bar=500 μm, white scale bar=50 μm.

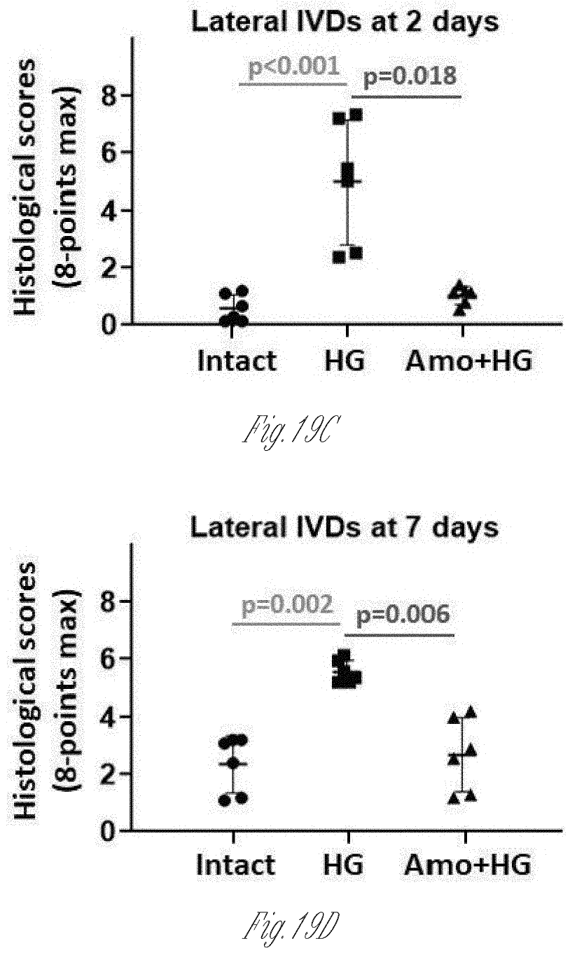

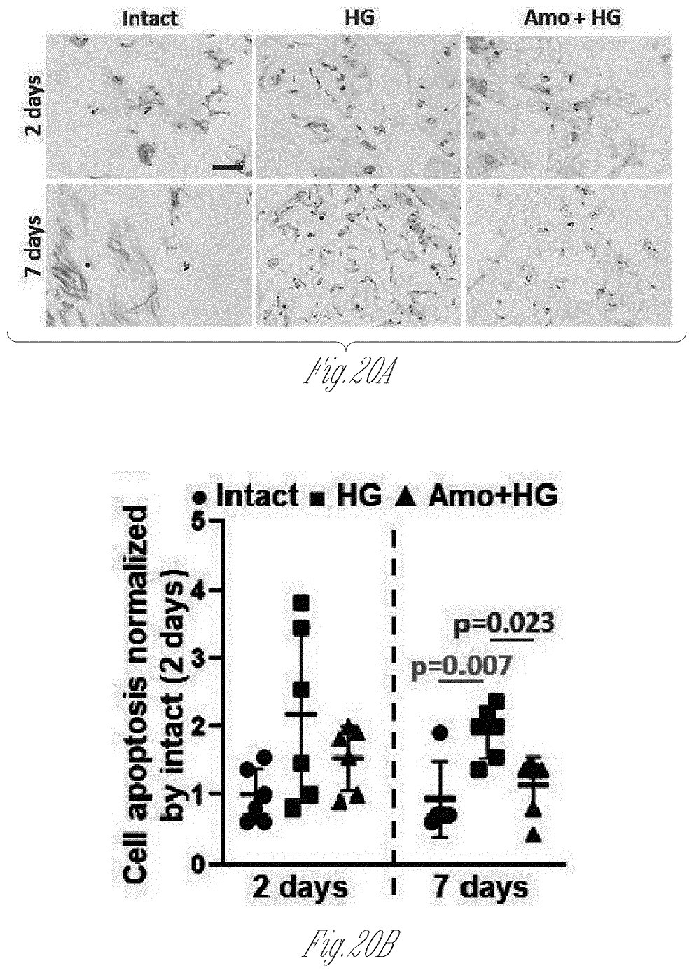

FIGS. 19A-19D. Histological examination of disc degeneration in the lateral intervertebral discs (IVDs). Extracellular matrix (ECM: upper panel) and cellular (lower panel) changes at (A) 2 and (B) 7 days. Blue arrowheads: clustered cells, yellow arrow bars: fibrous ECM, white asterisks: migrated endplate chondrocytes. (C) Histologic grading at 2 days (n=6). (D) Histologic grading at 6 days (n=6). Intact; no needle puncture with no injection, HG: hydrogel, Amo+HG: amobarbital in hydrogel. Black scale bar=500 μm, white scale bar=50 μm.

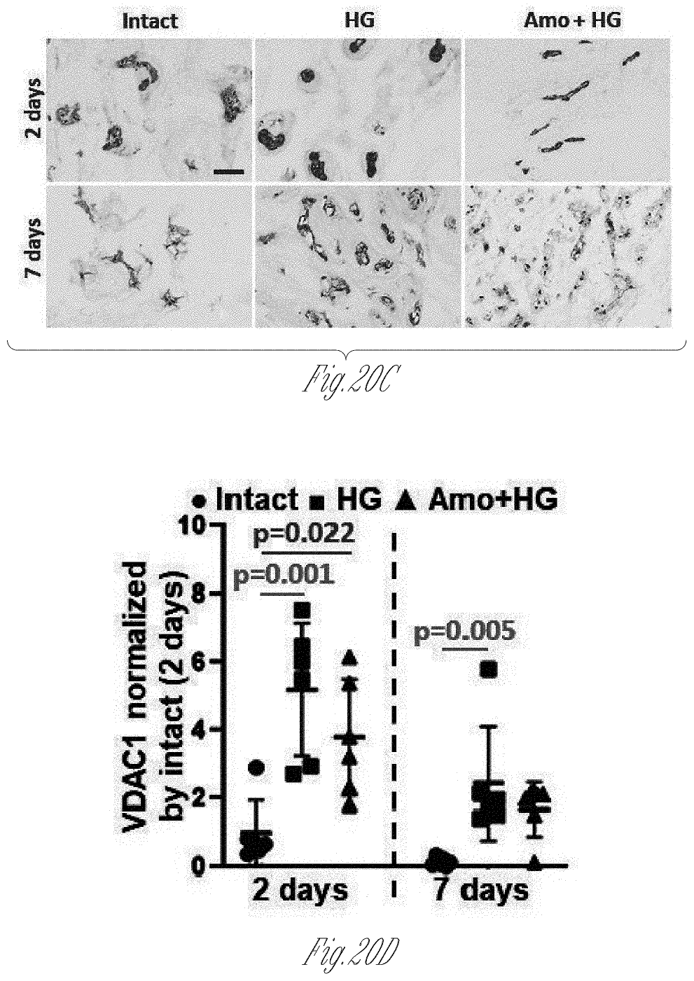

FIGS. 20A-20D. Nucleus pulposus cell apoptosis. (A) Representative images of Terminal deoxynucleotidyl transferase dUTP nick end labelling (TUNEL) staining at 2 and 7 days. (B) Quantified TUNEL-positive (brown) cells (n=5-6). (C) Representative images of voltage-dependent anion channel 1 (VDAC1: brown). (D) Quantified VDAC1 expression (mean grey intensity value/the number of nuclei) (n=6). Intact: no needle puncture with no injection, HG: hydrogel, Amo+HG: amobarbital in hydrogel. Blue: nuclei. Scale bar=50 μm.

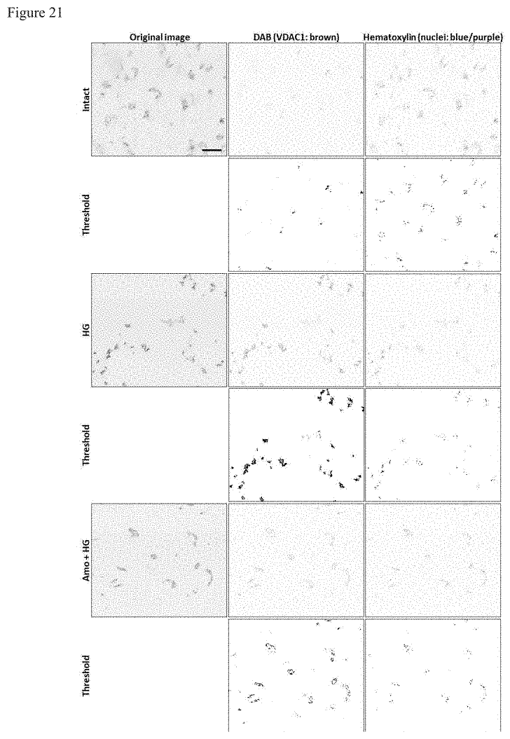

FIG. 21. Semi-quantification procedure of Voltage-dependent anion channel 1 (VDAC1) immunohistochemical staining using ImageJ Fiji software. Color deconvolution: H DAB, Maximal threshold of 3,3′-diaminobenzidine (DAB): 196, Maximal threshold of hematoxylin: 170, minimal nuclei size: 5 pixels. Scale bar=100 μm.

### DETAILED DESCRIPTION

### Definitions

“Hydrogel” as used herein means a water insoluble, naturally or chemically-induced cross-linked, three-dimensional network of polymer chains plus water that fills the voids between polymer.

“Intervertebral disc” is a fibrocartilageous tissue and consists of nucleus pulposus, annulus fibrosus, and endplate. The endplate covers the top and bottom of the disc and the central fibers of the inner two-third of the anulus fibrosus attach directly to the cartilaginous endplate. Nucleus pulposus is the jelly-like substance in the middle of the intervertebral disc. It functions to distribute hydraulic pressure in all directions within each disc under compressive loads. The nucleus pulposus includes water (70-90%), nucleus matrix (collagen fibrils, proteoglycan, and aggrecans) and nucleus pulposus cells.

“Intervertebral disc degeneration” is clinically considered as a significant source of low back pain which is one of the common problems in society and has ranked the second most common medical symptom leading to physician visits, hospitalization, and utilization of other health care services. Disc degeneration can be induced by a variety of factors such as genetic factors, limited nutrient supply, and mechanical stimulation.

### Introduction

Reactive oxygen species (ROS) are known as a major cause of cellular oxidative stress and complex I is one of the main contributors to superoxide production by mitochondria in mammalian cells (Hirst et al., 2008). Therefore, blocking complex I might decrease ROS production and attenuate mitochondrial damage resulting from intracellular ROS production. Amobarbital has been used in humans for years as a hypnotic, sedative, and anticonvulsant drug and is also a reversible inhibitor of mitochondrial electron transport complex I. It has been shown to successfully attenuate cell death in an ischemia reperfusion model (Aldakkak et al., 208; Stewart et al., 2009). In a previous study, amobarbital encapsulated in hydrogel was injected in a minipig hock intra-articular fracture model to prevent post-traumatic osteoarthritis (PTOA) (Coleman et al., 2018). Moreover, it significantly reduced complex I activity after blunt impact injuries in a bovine osteochondral explant model.

Mitogen-activated protein kinase (MAPK) appears to be important to oxidative stress in disc degeneration (Feng et al., 2017). In rat annulus fibrosus (AF) cells, oxidative stress activated MAPK signaling molecules, especially extracellular signal-regulated kinase (ERK), c-JUN N-terminal kinase (JNK), and p38, to regulate matrix metabolism and proinflammatory phenotype (Suzuki et al., 2015). Besides MAPK, the nuclear factor (erythroid-derived 2)-like 2 (Nrf2) is translocated into the nucleus where it links to the antioxidant-response element (ARE) signaling pathway, and plays as a multiorgan protector against oxidative stress (Mahmoud et al., 2017; Nguyen et al., 2009). Therefore, targeting these signaling pathways can be an attractive therapeutic strategy for disc degeneration prevention.

### Exemplary Compositions and Methods

Evidence that oxidative stress contributes to the progression of post-traumatic intervertebral disc (IVD) degeneration (IDD) suggests targeting oxidant metabolism in disc cells as a strategy to mitigate degeneration in injured discs. Amobarbital, a drug that suppresses mitochondrial activity, as may be a promising candidate for this purpose.

As described below, the preventive effects of amobarbital (Amo) on the progression of disc degeneration was assessed in ex vivo organ culture of rabbit spinal motion segments. A total of 36 rabbit thoracic and lumbar motion segments (T11/L2) including two vertebral bodies and one IVD were obtained from New Zealand White rabbits. The discs were punctured using a 20-gauge needle, and the hydrogel with or without Amo was injected into the injured site. A modified histological classification was applied to evaluate for IVD degenerative changes through Weigert's iron hematoxylin/Fast Green/Safranin-O staining. NP cell apoptosis was analyzed by terminal deoxynucleotidyl transferase dUTP nick end labelling (TUNEL) and voltage-dependent anion channel 1 (VDAC1) stains. An Amo/hydrogel injection allowed uniform distribution in the whole NP and showed sustained release for 3-4 days. Amo treatment after a discal injury prohibited morphologic changes of NP notochordal cells, structural changes of extracellular matrix, endplate chondrocyte migration, and cell apoptosis compared with the hydrogel only group. The Amo injection loaded in a temperature-sensitive hydrogel prohibited cellular and structural disc changes in NP cells during ex vivo organ culture of rabbit spine motion segments with a disc puncture. Therefore, Amo treatment targeting oxidative stress may prevent degenerative disc degeneration.

As disclosed herein, a stable organ culture system was established for both intact and punctured rabbit intervertebral discs. This system is beneficial to evaluate the effect of amobarbital and to determine delivery strategy before in vivo animal studies. A drug delivery system is useful for efficient and safe local drug delivery in the intervertebral disc. In one embodiment, local delivery vehicles were prepared including a temperature-responsive in situ-forming hydrogel, pellet for extended-release of drug, and microparticles. In particular, Pluronics® F-127/hyaluronic acid-based hydrogel can allow the local injection into the targeted nucleus pulposus without any loss of matrix via back-flow.

Thus, amobarbital when incorporated into thermoresponsive hydrogel for efficient delivery and sustained release can prevent oxidative damage to disc cells and extracellular matrix (ECM) from acute intervertebral disc injuries, which eventually lead to inhibit or treat disc degeneration.

Thus, a therapeutic strategy was developed for minimally-invasive, local delivery of controlled-released amobarbital via a temperature-sensitive hydrogel to prevent, inhibit or treat oxidative stress in the intervertebral disc from traumatic injuries. The present in vitro data indicate that pre- and post-treatment with amobarbital prevented disc cell apoptosis and mitochondrial dysfunction such as excessive superoxide, declined membrane potential, and increased basal oxygen consumption rate via mitogen-activated protein kinase (MAPK; phosphorylation of ERK, JNK, and p38) and nuclear factor (erythroid-derived 2)-like 2 (Nrf2) signaling pathways. These findings suggest that amobarbital and functionally similar and/or structurally similar agents represent a therapeutic option to protect oxidative stress in disc cells and eventually to prevent intervertebral disc degeneration.

### Compositions and Methods to Prevent, Inhibit or Treat Spinal Disease

The present compositions and methods are useful to prevent, inhibit or treat spinal disease, optionally resulting from injury. In one embodiment, the compositions employed in the method are hydrogels. Hydrogels can be classified as those with crosslinked networks having permanent junctions or those with physical networks having transient junctions arising from polymer chain entanglements or physical interactions, e.g., ionic interactions, hydrogen bonds or hydrophobic interactions. Natural materials useful in hydrogels include natural polymers, which are biocompatible, biodegradable, support cellular activities, and may include proteins like fibrin, collagen or gelatin, and/or polysaccharides like hyaluronic acid, starch, alginate or agarose. Synthetic polymers useful in hydrogels are prepared by chemical polymerization and include by way of example poloxamers, acrylic acid, hydroxyethyl-methacrylate (HEMA), vinyl acetate, and methacrylic acid (MAA).

Various methods may be used to prepare hydrogels, e.g., crosslinkers, copolymerization of monomers using multifunctional co-monomer, cross linking of linear polymers by irradiation or by chemical compounds. Monomers contain an ionizable group that can be ionized or can undergo a substitution reaction after the polymerization is completed. Exemplary crosslinkers are glutaraldehyde, calcium chloride, and oxidized konjac glucomannan (DAK).

Some classes of hydrogels include (a) homopolymeric hydrogels which are derived from a single species of monomer. Homopolymers may have cross-linked skeletal structure depending on the nature of the monomer and polymerization technique; (b) copolymeric hydrogels which are comprised of two or more different monomer species with at least one hydrophilic component, arranged in a random, block or alternating configuration along the chain of the polymer network; (c) multipolymer interpenetrating polymeric hydrogel (IPN) which is made of two independent cross-linked synthetic and/or natural polymer components, contained in a network form. In semi-IPN hydrogel, one component is a cross-linked polymer and other component is a non-cross-linked polymer.

Biodegradable hydrogels as a delivery vehicle have the advantage of being environmentally friendly to the human body (due to their biodegradability) and of providing more predictable, controlled release of the impregnated drugs. Hydrogels are of special interest in biological environments since they have high water content as is found in body tissue and are highly biocompatible. Hydrogels and natural biological gels have hydrodynamic properties similar to that of cells and tissues. Hydrogels minimize mechanical and frictional irritation to the surrounding tissue because of their soft and compliant nature. Therefore, hydrogels provide a far more user-friendly delivery vehicle than the relatively hydrophobic carriers like silicone, or vinyl acetate.

Biocompatible materials that may be present in a hydrogel include, e.g., permeable configurations or morphologies, such as polyvinyl alcohol, polyvinylpyrrolidone and polyacrylamide, polyethylene oxide, poly(2-hydroxyethyl methacrylate); natural polymers such as polysaccharides, gums and starches; and include poly[α(4-aminobutyl)]-1-glycolic acid, polyethylene oxide, polyorthoesters, silk-elastin-like polymers, alginate, poly(ethylene-co-vinyl acetate) (EVA), microspheres such as poly (D, L-lactide-co-glycolide) copolymer and poly(L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, or agarose.

In one embodiment, the hydrogel includes poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, the hydrogel includes collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PDS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as alginate, agarose, starch, fibrin, collagen, gelatin, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the hydrogel comprises a poloxamer (polyoxyethylene, polyoxypropylene block copolymers. e.g., poloxamer 127, 231, 182 or 184).

### Exemplary Components for Use in Hydrogels

In one embodiment, the hydrogels useful in the compositions and methods of the invention are synthesized from a naturally occurring biodegradable, biocompatible, and hydrophilic polysaccharide, and optionally a synthetic biocompatible polymer, such as poloxamers, polylactide (PLA), polyglycolide (PGA), or poly(lactic acid co-glycolic acid) (PLGA).

The composition that forms a hydrogel, e.g., a reverse temperature-sensitive hydrogel, includes a polysaccharide, including chemically cross-linked polysaccharides and a synthetic or natural polymer, and a compound that reversibly inhibits complex I. One exemplary polysaccharide is hyaluronic acid, a naturally occurring copolymer composed of the sugars, glucuronic acid and N-acetylglucosamine. Specifically, hyaluronic acid, also named hyaluronan or sodium hyaluronate, is a high molecular weight (10⁵-10⁷ Da) naturally occurring biodegradable polymer that is an unbranched non-sulfated glycosaminoglycan (GAG) composed of repeating disaccharides (β-1,4-D-glucuronic acid (known as uronic acid) and β-1,3-N-acetyl-D-glucosamide). Hyaluronic acid has an average molecular weight of 4-5 MDa Hyaluronic acid can include several thousand sugar molecules in the backbone. Hyaluronic acid is a polyanion that can self-associate and that can also bind to water molecules (when not bound to other molecules) giving it a stiff, viscous quality similar to gelatin. Hylans are cross-linked hyaluronan chains in which the carboxylic and N-acetyl groups are unaffected. The molecular weight of hylan A is about 6 million Da. Hylans can be water insoluble as a gel (e.g., hylan B).

Hyaluronic acid's characteristics include its consistency, biocompatibility, hydrophilicity, viscoelasticity and limited immunogenicity. The hyaluronic acid backbone is stiffened in physiological solution via a combination of internal hydrogen bonds, interactions with solvents, and the chemical structure of the disaccharide. At very low concentrations, hyaluronic acid chains entangle each other, leading to a mild viscosity (molecular weight dependent). On the other hand, hyaluronic acid solutions at higher concentrations have a higher than expected viscosity due to greater hyaluronic acid chain entanglement that is shear-dependent. Thus, solutions containing hyaluronic acid are viscous, but the viscosity is tunable by varying hyaluronic acid concentration and the amount of cross-linking. In addition to the unique viscosity of hyaluronic acid, the viscoelasticity of hyaluronic acid is another characteristic resulting from entanglement and self-association of hyaluronic acid random coils in solution. Viscoelasticity of hyaluronic acid can be tied to molecular interactions which are also dependent on concentration and molecular weight.

Exemplary hyaluronic acid solutions for injection are shown in Table 1, and include Synvisc® (high molecular weight hyaluronic acid due to crosslinking), Hyalgan® (sodium hyaluronate solution), and Orthovisc® (one of the viscosupplements with the highest hyaluronic acid concentration, which has lower viscosity than Synvisc®) (the properties of those are shown in Table 2).

TABLE 1

| Brand name (Generic name) | Molecular weight (kDa) |
|---|---|
| Durolane ® (Hyaluronic acid, 2%) | 1000 |
| Fermathron ® (Sodium hyaluronate, 1%) | 1000 |
| Hyalgan ® (Sodium hyaluronate, 1%) | 500-730 |
| NeoVisc ® (Sodium hyaluronate, 1%) | 1000 |
| Orthovisc ® (Sodium hyaluronate, 1%) | 1000-2900 |
| Ostenil ® (Sodium hyaluronate, 1%) | 1000-2000 |
| Supartz ® (Sodium hyaluronate, 1%) | 620-1170 |
| Suplasyn ® (Sodium hyaluronate, 1%) | 500-730 |
| Synvisc ® (Hylan G-F 20; Crosslinked HA) | 6000-7000 |
| Gel-One ® (Cross-linked hyaluronate, 1%) | N.A. |

TABLE 2

| | | Viscoelastic properties | |
|---|---|---|---|
| | Molecular | Elastic | Viscous |
| | weight | modulus (G′) | modulus (G″) |
| Brand name | (kDa) | (Pa) at 2.5 Hz | (Pa) at 2.5 Hz |
| Hyalgan ® (Uncrosslinked) | 500-730 | 0.6 | 3 |
| Orthovisc ® (Uncrosslinked) | 1000-2900 | 60 | 46 |
| Synvisc ® (Crosslinked polymer) | 6000-7000 | 111 ± 13 | 25 ± 2 |

Dextran is another polysaccharide and is formed primarily of 1,6-α-D-glucopyranosyl residues and has three hydroxyl groups per glucose residue that could provide greater flexibility in the formulation of hydrogels. Dextran has been widely used for many biomedical purposes, such as plasma expander and controlled drug delivery vehicle, because of its highly hydrophilic nature and biocompatibility. It is also possible to incorporate dextranase in order to facilitate biodegradation of dextran for the meeting of specific clinical needs.

In one embodiment, the hydrogel comprises a poloxamer. Poloxamers are nonionic triblock copolymers composed of a central hydrophobic chain of polyoxypropylene (poly(propylene oxide)) flanked by two hydrophilic chains of polyoxyethylene (poly(ethylene oxide)) (α-Hydro-ω-hydroxypoly (oxyethylene)ₐ poly (ocypropylene)_{b} poly (olxyethylene)ₐ block copolymer, with two hydrophilic chains of ethylene oxide chains (PEO) that sandwich one hydrophobic propylene oxide chain (PPO) giving a chemical formula HO(C₂H₄O)ₐ(C₃H₆O)_{b}(C₂H₄O)ₐ). For example, poloxamer 407 is a triblock copolymer consisting of a central hydrophobic block of polypropylene glycol flanked by two hydrophilic blocks of polyethylene glycol. Exemplary poloxamers include but are not limited to polyethylene-propylene glycol copolymer, e.g., Supronic, Pluronic or Tetronic a non-ionic triblock copolymer.

The common representation of Poloxamer is indicated as ‘P’ succeeded by three digits where the first two digits are to be multiplied by 100 and that gives the molecular mass of the hydrophobic propylene oxide and the last digit is to be multiplied by ten that gives the content of hydrophilic ethylene oxide in percentage. Poloxamers usually have an efficient thermoreversible property with characteristics sol-gel transition temperature. Below the transition temperature it is present as a solution and above this temperature the solution results in interaction of the copolymer segment which leads to gelation. Poloxamers are non-toxic and non-irritant.

TABLE 3

| | | | | | Average | | |
|---|---|---|---|---|---|---|---|
| | | | Ethylene | | molecular | Weight % of | |
| | | | oxide | Propylene | mass | Oxyethylene | |
| | | Physical | units (n)^{a} | oxide units (n)^{a} | PhEur 2005; | PhEur | USPNF |
| Poloxamer | Pluronic | form | (a) | (b) | USPNF 23 | 2005 | 23 |
| 124 | L44 | Liquid | 10-15 | 18-23 | 2090-2360 | 44.8-48.6 | 46.7 ± 1.9 |
| 188 | F68 | Solid | 75-85 | 25-40 | 7680-9510 | 79.9-83.7 | 81.8 + 1.9 |
| 237 | F87 | Solid | 60-68 | 35-40 | 6840-8830 | 70.5-74.3 | 72.4 ± 1.9 |
| 338 | F108 | Solid | 137-146 | 42-47 | 12700-17400 | 81.4-84.9 | 83.1 ± 1.7 |
| 407 | F127 | Solid | 95-105 | 54-60 | 9840-14600 | 71.5-74.9 | 73.2 ± 1.7 |

Compounds that reversibly inhibit complex I include but are not limited to amobarbital or derivatives thereof, metformin or derivatives thereof, or adenosine diphosphate ribose analogs that disrupt NADH binding. However, non-reversible inhibitors of complex I, e.g., Rotenone. Piericidin A or Rolliniastatin 1 and 2, in low doses, may also have some benefit to cartilage after injury as a result of altering ROS.

For example, an injectable temperature-sensitive hydrogel (e.g., one having hyaluronic acid, such as Gel One which is chemically cross-linked and has a high molecular weight, and optionally has a poloxamer, such as F68 or F127) is employed to deliver a therapeutic agent, for instance, the hydrogel is loaded with amobarbital. The hydrogel becomes firm once injected (e.g., preventing leakage from a disc) allowing the therapeutic to be retained in disc, for example, for about 3 days after injection. In one embodiment, the hydrogel comprises 17% (w/v) F-127 and 0.2% (w/v) hyaluronic acid, and is loaded with 2.5 mM amobarbital.

### Formulations and Dosages

The components of the composition can be formulated as pharmaceutical compositions and administered to a mammalian host, such as a human patient in a variety of forms adapted to the chosen route of administration. In one embodiment, the components of the composition are locally administered to a site of cartilage damage or suspected cartilage damage, or is administered prophylactically.

In one embodiment, the components of the composition may be administered by infusion or injection. Solutions may be prepared in water, optionally mixed with a nontoxic surfactant. Dispersions may also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride.

Sterile injectable solutions may be prepared by incorporating the active agent in the required amount in the appropriate solvent with various other ingredients, as required, optionally followed by filter sterilization. In the case of sterile powders for the preparation of sterile injectable solutions, the methods of preparation include vacuum drying and the freeze-drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compound(s) in the composition can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the therapeutic compound(s) in a composition, may be from about 0.1-25% wt/vol, e.g., from about 0.5-10% wt/vol. The concentration in a semi-solid or solid composition such as a gel or a powder may be about 0.1-5% wt/vol, e.g., about 0.5-2.5% wt/vol.

The amount of the compound for use alone or with other agents may vary with the type of hydrogel, route of administration, the nature of the condition being treated and the age and condition of the patient, and will be ultimately at the discretion of the attendant physician or clinician.

The components of the composition may be conveniently administered in unit dosage form; for example, containing 5 to 1000 mg, conveniently 10 to 750 mg, or conveniently 50 to 500 mg of active ingredient per unit dosage form

In general, however, a suitable dose may be in the range of from about 0.5 to about 100 mg/kg, e.g., from about 10 to about 75 mg/kg of body weight per day, such as 3 to about 50 mg per kilogram body weight of the recipient, for example in the range of 6 to 90 mg/kg, e.g., in the range of 15 to 60 mg/kg.

### EXEMPLARY EMBODIMENTS

The disclosure provides for a method to prevent, inhibit or treat intervertebral disc disease in a mammal, comprising locally administering to a disc of the mammal an effective amount of a hydrogel composition comprising hyaluronic acid and Pluronics® (poloxamer), and an amount of amobarbital or a derivative thereof effective to prevent, inhibit or treat intervertebral disc degeneration. In one embodiment, the composition is injected. In one embodiment, the hyaluronic acid is about or greater than 0.5 MDa. In one embodiment, the hyaluronic acid is present in the composition from about 0.01% (wt/vol) and up to about 2.0% (wt/vol). In one embodiment, the hyaluronic acid is present at about 0.2% (wt/vol) and up to about 1.0% (wt/vol). In one embodiment, the Pluronics® F127 (poloxamer 407 or P407) is present in the composition from about 15% (wt/vol) and up to about 20% (wt/vol). In one embodiment, the hydrogel further comprises N-isopropyl acrylamide polymer, a poly saccharide other than hyaluronic acid, hydroxypropylcellulose, karya gum, guar gum, gellan gum, alginate, ethyl-droxyethylcellulose, poly(ethyleneoxide-b-propylene oxide-b-ethylene oxide), a Pluronics® polymer, a poly(ethylene glycol)/poly(D,L-lactic acid-co-glycolic acid) block copolymer, a polyphosphazine, a polyacrylate, a Tetronics™ polymer, or a poly(ethylene oxide)-poly(propylene) glycol block copolymer. In one embodiment, the composition comprises amobarbital, pentobarbital, secobarbital, phenobarbital, adenosine diphosphate ribose, or metformin, or a derivative thereof. In one embodiment, the amount inhibits mitochondrial dysfunction, disc cell energy dysfunction, or disc cell death. In one embodiment, the amobarbital or a derivative thereof prevents the formation of mitochondrial oxidants or stimulates glycolytic ATP production. In one embodiment, disc degeneration is inhibited. In one embodiment, the disc is a thoracic disc. In one embodiment, the disc is a lumbar disc. In one embodiment, the disc is a cervical disc. In one embodiment, the administration reduces reactive oxygen species (ROS) production in the nucleus pulposus. In one embodiment, the administration is within 4 days of spinal injury or surgery. In one embodiment, the administration is within 5 to 12 hours of spinal injury or surgery. In one embodiment, the mammal is a human. In one embodiment, the composition is a thermoresponsive or temperature sensitive hydrogel. In one embodiment, the mammal has an injury in the nucleus pulposus, annulus fibrosus, or endplate. In one embodiment, the mammal has disc herniation. In one embodiment, a syringe is employed to administer the composition. In one embodiment, the syringe has a 22 to 24-gauge needle.

Also provided is a method to prevent, inhibit or treat spinal degeneration in a mammal, comprising locally administering to a spine of the mammal an effective amount of a hydrogel composition comprising hyaluronic acid, hydroxypropylcellulose, karaya gum (KG), guar gum (GUG), or gellan gum (GEG) and a compound in an amount that reversibly inhibits respiratory enzyme complex, and optionally a synthetic polymer. In one embodiment, the composition is injected. In one embodiment, the composition comprises hyaluronic acid and a synthetic polymer comprising a poloxamer. In one embodiment, the composition comprises F127 or F68. In one embodiment, the composition comprises amobarbital, pentobarbital, secobarbital, phenobarbital, barbital, adenosine diphosphate ribose, or metformin, or a derivative thereof. In one embodiment, disc degeneration is inhibited. In one embodiment, the disc is a thoracic disc. In one embodiment, the disc is a lumbar disc. In one embodiment, the disc is a cervical disc. In one embodiment, the administration reduces ROS production in the nucleus pulposus. In one embodiment, the administration is within 4 days of spinal injury or surgery. In one embodiment, the administration is within 5 to 12 hours of spinal injury or surgery. In one embodiment, the mammal is a human. In one embodiment, the composition is a temperature sensitive hydrogel. In one embodiment, the composition comprises hyaluronic acid. In one embodiment, the hyaluronic acid is about or greater than 0.5 M Dalton or about or greater than 1.0 M Dalton. In one embodiment, the hyaluronic acid is present in the composition from about 0.01% (wt/vol) and up to about 2.0% (wt/vol). In one embodiment, the hyaluronic acid is present at about 0.2% wt/vol to about 1.0% wt/vol.

Further provided is a method to prevent, inhibit or treat intervertebral disc degeneration in a mammal, comprising injecting an intervertebral disc of the mammal with an effective amount of a hydrogel composition comprising hyaluronic acid and a compound in an amount that reversibly inhibits respiratory enzyme complex, and optionally a synthetic polymer. In one embodiment, the mammal is a human. In one embodiment, the disc is herniated. In one embodiment, a syringe is employed to administer the composition. In one embodiment, the syringe has a 22-gauge needle. In one embodiment, the disc is a thoracic disc. In one embodiment, the disc is a lumbar disc.

The invention will be described by the following non-limiting examples.

### Example 1

### Materials and Methods

### Study Design

The protective effects of amobarbital were evaluated using rabbit nucleus pulposus cells and tissues (FIG. 1). NAC, which is a well-known antioxidant, was used for control to compare with amobarbital. In nucleus pulposus cell culture study, the cells were pre-treated with amobarbital, and then oxidative stress was induced by tert-butyl hydrogen peroxide (tBHP). In order to evaluate the therapeutic effect of amobarbital in nucleus pulposus tissues, oxidative stress was induced by traumatic injury, and then injured tissues were treated with amobarbital. Antioxidative effects of amobarbital were evaluated by cell apoptosis, ROS production, mitochondrial membrane potential, and signaling pathways.

### Isolation of Nucleus Pulposus Cells

A total of 20 rabbit lumbar and thoracic intervertebral discs were obtained from 5 young adult rabbit cadavers (New Zealand White; 3-4 kg) without radiographic evidence of trauma. Under sterile conditions, the intervertebral discs were dissected by removing the posterior elements and soft tissues. The nucleus pulposus tissues were harvested and digested with 0.25% trypsin for 20 minutes (min) and 0.2% collagenase type I for 5 hours (h) to isolate nucleus pulposus cells. The cells were cultured in Dulbecco's modified eagle medium/nutrient mixture F-12 (DMEM/F-12; Gibco) supplemented with 10% fetal bovine serum (Thermo Fisher Scientific, Waltham, Mass., USA), 50 μg/ml L-ascorbate, 100 U/ml penicillin-streptomycin (Thermo Fisher Scientific), and 2.5 μg/ml amphotericin B (Sigma-Aldrich. St. Louis, Mo., USA) in physiological culture condition (5% O₂/CO₂ at 37° C.).

Cytotoxicity of tBHP, NAC, and Amobarbital

The nucleus pulposus cells were treated with various concentrations of tBHP (25, 50, 100, 200, or 400 μM) (Sigma-Aldrich) for 1 h, NAC (2, 10, 50, or 250 mM) (Sigma-Aldrich) for 2 h, and amobarbital (0.5, 2.5, 12.5 or 62.5 mM) (Amytal® sodium; Bausch Health, Bridgewater, N.J., USA) for 2 h. The cells were washed after the treatment, and cytotoxicity was determined by CellTiter 96® Aqueous One Solution (Promega, Madison, Wis., USA). Twenty μl of One Solution Reagent was added into each well of the 96-well plates containing 100 μl of medium and the plate was incubated at 37° C. in a humidified incubator for 2 h. The absorbance was recorded at 490 nm using a 96-well plate reader (SpectraMax M5, Molecular Devices, San Jose, Calif., USA). Additionally, cells were pre-treated with NAC (0.1, 2, or 10 mM) or amobarbital (0.1, 0.5, or 2.5 mM) for 2 h prior to tBHP exposure for 1 h to determine the optimal concentration.

### Induction of Oxidative Stress in Nucleus Pulposus Cells and Amobarbital Treatment

The nucleus pulposus cells (passage 2) were seeded at a density of 1×10⁴ in 96-well plates and 2×10⁵ in 6-well plates for in vitro assays and immunofluorescence (IF) staining, respectively. When the cells reached 80% confluence, they were pre-treated with 10 mM NAC and/or 2.5 mM amobarbital for 2 h. After washing, 50 μM tBHP was added to induce cellular oxidative stress for 1 h (FIG. 1a).

### Anti-Apoptotic Effect of Amobarbital

Alexa Flour 488® Annexin V/Dead Cell Apoptosis Kit (Thermo Fisher Scientific) containing Annexin V and propidium iodine (PI) was used to evaluate anti-apoptotic effect of amobarbital. After tBHP exposure with pre-treatment of NAC and amobarbital, nucleus pulposus cells were washed twice with cold phosphate buffered saline (PBS; Thermo Fisher Scientific) and incubated with Annexin V (25 mM HEPES, 140 mM NaCl, 1 mM EDTA, and 0.1% bovine serum albumin; 1:20 dilution), 2 μg/ml PI, and 2 μg/ml 4′,6-diamidino-2-phenylindole (DAPI; Thermo Fisher Scientific) at room temperature for 15 min. The cells were washed twice in 1× binding buffer and imaged by an Olympus FV1000 confocal microscope (Olympus, Center Valley, Pa., USA). Apoptotic (Annexin V positive), necrotic (both Annexin V and PI positive), and live (DAPI positive) cells were quantified using ImageJ software (NIH, Bethesda, Md., USA).

### Mitochondrial ROS Production

Nucleus pulposus cells were stained with 5 μM MitoSOX Red (Invitrogen, Eugene, Oreg., USA) for mitochondrial ROS measurement at 37° C. in the dark for 10 min. This oxidation sensitive dye provides an indication of ROS production in mitochondria, and the majority of MitoSOX Red staining is believed to be superoxide anion. After washing, the cells were fixed with 4% paraformaldehyde (Sigma-Aldrich) and counterstained with DAPI. Fluorescent images were obtained using an Olympus FV1000 confocal microscope. For the quantification, the cells were prepared in a 96-well black plate (Thermo Fisher Scientific) and the fluorescence was measured using a SpectraMax Spectrofluorometer (Molecular Devices) at 510/580 nm (excitation/emission).

### Mitochondrial Membrane Potential

Mitochondrial membrane potential was determined using a JC-1 Assay Kit (Cayman Chemical. Ann Arbor, Mich., USA) following the manufacturer's instructions. The cells were stained with JC-1 Staining Solution (1:10 dilution) at 37° C. in the dark for 30 min. After centrifuging, the plate was run at both 530/595 nm (excitation/emission; J-aggregates) and 485/535 nm (excitation/emission; monomers) in a SpectraMax Spectrofluorometer. The ratio of fluorescence intensity was calculated by J-aggregates (healthy mitochondria)/monomers (unhealthy mitochondria) and normalized by viable cells.

### Immunofluorescence (IF) Staining for Signaling Pathways

The nucleus pulposus cells were processed for IF staining for MAPKs including ERK, JNK, and p38. PD98059 (20 μM). SB203580 (20 μM), and SP600125 (20 μM) (all from Sigma-Aldrich) were used as ERK, JNK, and p38 inhibitors, respectively (Li et al., 2016). Briefly, the cells were fixed with 4% paraformaldehyde for 10 min. permeabilized with 0.2% Triton X-100 for 10 min. and blocked with 10% goat serum for 1 h. After washing, the cells were incubated with the primary antibodies against phosphorylated anti-ERK (1:200 dilution; Cell Signaling Technology, Danvers, Mass. USA), phosphorylated anti-JNK (1:200 dilution; Cell Signaling Technology), and phosphorylated anti-p38 (1:200 dilution; Cell Signaling Technology) at 4° C. overnight. Goat anti-mouse IgG (1:500 dilution; Cell Signaling Technology) and DAPI were used for secondary antibody and counterstain, respectively. Fluorescent images were obtained using an Olympus FV1000 confocal microscope.

### Oxidative Stress Endpoints in Ex Vivo Organ Culture of Intervertebral Discs

A total of 12 lumbar spine motion segments consisting of 2 vertebrae and an intervertebral disc were dissected, and oxidative stress was induced by traumatically transverse cutting of the discs (FIG. 1b). After washing, the tissues were immediately treated with 10 mM NAC or 2.5 mM amobarbital for 24 h. They were then stained with dihydroethidium (DHE; 1:500 dilution; Thermo Fisher Scientific) to visualize ROS production with Calcein AM (1:1,000 dilution; Thermo Fisher Scientific) as a counterstain for live cells. Another set of the intervertebral discs was stained for Nrf2 with mouse anti-Nrf2 primary antibody (1:200; Abcam. Cambridge, Mass., USA), and goat anti-mouse IgG secondary antibody (1:500 dilution; Cell Signaling Technology), and DAPI.

### Statistics

All quantified data were normalized by control (no treatment of tBHP) and expressed in percentages. The bar graphs were expressed as the mean values with the standard deviation. Data were compared by one-way ANOVA with the Tukey post-hoc test using SPSS Statistics (Version 25; IBM, Armonk, N.Y., USA). Statistical significance was set at p<0.05.

### Results

Determination of Optimal Concentration of tBHP, NAC, and Amobarbital

Using nucleus pulposus cells, the cytotoxicity of varying concentrations of tBHP. NAC, and amobarbital was measured. The results showed a significant decrease of cell viability at higher than 25 μM tBHP (90.8% at 25 μM and 77.1% at 50 μM; p<0.001 vs. control) (FIG. 2a). The cell loss was similar at the range of 100-400 μM tBHP (70.6-72.9%). In order to evaluate the anti-apoptotic effect of amobarbital, 50 μM tBHP exposure for 1 h was selected for all in vitro studies. Cells treated with NAC or amobarbital for 2 h exhibited no cytotoxicity at lower than 10 mM and 2.5 mM, respectively (FIG. 2a). The protective effect of NAC and amobarbital on cell viability was assessed immediately (FIG. 2b) and at 24 h (FIG. 2c) after pre-treatment prior to 50 μM tBHP exposure. NAC and amobarbital significantly prohibited the cell death at 0.1-10 mM (p<0.001 vs. control) until 24 h and 2.5 mM at only 0 h (p<0.001 vs. control), respectively. Based on these results, 10 mM NAC and 2.5 mM amobarbital were used for the following studies.

### Anti-Apoptotic Effect of Amobarbital

Nucleus pulposus cells showed apparent positive staining of Annexin V and PI, indicating apoptotic (Annexin V positive) and necrotic (both Annexin V and PI positive) cells when exposed to 50 μM tBHP (FIG. 2e). In contrast, the number of damaged cells was dramatically decreased in both NAC and amobarbital. In the quantified data, amobarbital treatment showed statistically significant increase of live cells (92.2% in amobarbital only and 91.7% in NAC and amobarbital mixture) with approximately 8% apoptotic and necrotic cells (p<0.001 vs. control) (FIG. 2d).

Protective Effect of Amobarbital from Mitochondrial Damage

Nucleus pulposus cells treated with tBHP had more ROS production in the mitochondria compared with control as imaged by MitoSOX Red staining (FIG. 3a). Pre-treatment with NAC and to an even greater degree amobarbital led to a large decrease of MitoSOX-stained cells. Similarly, NAC and amobarbital protected mitochondrial damage with approximately 50% decrease of MitoSOX oxidation compared with tBHP in fluorescence measurement (p<0.001 vs. tBHP) which was close to control (FIG. 3b). The effect of oxidative stress on mitochondrial membrane potential, which is an essential parameter of mitochondrial function, was evaluated using JC-1 assay. The ratio of J-aggregate form (healthy mitochondria) to monomeric form (unhealthy mitochondria) was significantly reduced in the group of tBHP (p<0.001 vs. control) (FIG. 3c). In contrast, NAC or amobarbital treatment enhanced the membrane potential (p=0.009 vs. tBHP and p=0.04 vs. tBHP). Thus, these data indicate that both NAC and amobarbital have potential for protection against tBHP-induced oxidative damage to mitochondrial function in nucleus pulposus cells.

### Signaling Pathways

In order to evaluate signaling pathway responses of amobarbital in nucleus pulposus cells, the phosphorylation of MAPK (ERK, JNK, and p38) pathways was examined by IF staining. The results showed that all signaling pathways of MAPK were significantly phosphorylated after 50 μM tBHP treatment for 1 h (FIG. 4). In particular, the rates of phosphorylated JNK and p38 were approximately 5.9 and 9.7 times in tBHP exposure compared with control, respectively (JNK: p=0.005 vs. control, p38: p<0.001 vs. control) (FIG. 4c-f). The pre-treatment of both NAC or amobarbital inhibited the phosphorylation of ERK (NAC: p=0.046 vs. tBHP, amobarbital: p=0.007 vs. tBHP), JNK (NAC: p=0.005 vs. tBHP, amobarbital: p=0.001 vs. tBHP), and p38 (NAC: p=0.001 vs. tBHP, amobarbital: p<0.001 vs. tBHP). Similarly, the inhibitors (ERK: 20 μM PD98059, JNK: 20 μM SB203580, p38: 20 μM SP600125) also minimized the phosphorylation of all MAPK signaling molecules.

### Therapeutic Effects of Amobarbital in Ex Vivo Organ Culture of Intervertebral Discs

The efficacy of amobarbital on traumatically injured intervertebral disc tissues was examined via ex vivo organ culture. The discs were transversely sliced, and the generation of oxidative stress in the nucleus pulposus tissues was confirmed by DHE staining (FIGS. 5a and b). Under oxidative stress, post-treatment of NAC or amobarbital dramatically reduced dye oxidation (p<0.001 vs. control), implicating superoxide in this pathology. As an indicator of oxidative stress associated with other injuries, Nrf2 expression and localization was evaluated. Injured intervertebral discs showed a much higher proportion of nuclear staining. The nucleus pulposus cells under oxidative stress by discal injury expressed Nrf2 in the nucleus, while the protected cells showed the majority of their staining within the cytoplasm (FIG. 5c). In a mild contrast, both NAC and amobarbital activated approximately 6.8 (p=0.002 vs. control) and 7.8 (p=0.005 vs. control) times higher Nrf2 expression in the cytosol compared with injured control group (FIG. 5d). This may indicate that Nrf2 regulation after disc injury is responsive to antioxidants in multiple ways; however, the clear difference in localization indicates that Nrf2 is fully active and translocated to the nucleus after injury and that this translocation is prevented with amobarbital or NAC. Thus, it was confirmed that amobarbital has therapeutic potential in blocking oxidative stress after acute discal injury.

### Discussion

The nucleus pulposus of intervertebral disc, which contains high water content and nucleus matrix (proteoglycan, aggrecan, and collagen), is essential to maintain biomechanical transmission of compressive loads. Since the nucleus pulposus becomes a more fibrous tissue due to the loss of proteoglycan and water with aging or traumatic injury, the tissue can be subjected to physical stress and eventually lead to disc degeneration (Vo et al., 2016). Oxidative stress is one of the contributors to induce disc degeneration due to nucleus pulposus cell apoptosis and matrix degradation (Feng et al., 2017). Several studies have shown that the evidence of oxidative stress was observed in human and animal degenerative discs, and promoted the expression of catabolic factors such as tumor necrosis factor-alpha (TNF-α) and matrix metalloprotease-3 (MMP-3) (Dimozi et al., 2015: Suzuki et al., 2015). Thus, targeting oxidative stress is a promising therapeutic approach to prevent disc degeneration.

In this study, the source of oxidants in disc injury was investigated by comparing the effects of NAC and amobarbital on the cytotoxicity, apoptosis, mitochondrial dysfunction of nucleus pulposus cells, as well as on the molecular mechanisms at play. NAC, which has been widely used as a ROS scavenger in disc degeneration studies, suppressed cell apoptosis. ROS production, excessive autophagy, and catabolic and proinflammatory phenotypes (Dimozi et al., 2015; Feng et al., 2017). The results showed that pretreatment with NAC considerably increased cell viability at concentrations ranging from 0.1 to 10 mM (FIGS. 2b and c) and decreased mitochondrial ROS production and membrane potential (FIG. 3a, b, and c), however, there was no significant difference in reduction of nucleus pulposus cell apoptosis (FIGS. 2d and e). On the other hand, amobarbital pre-treatment dramatically prohibited cell apoptosis & necrosis (FIGS. 2d and e) and mitochondrial dysfunction such as excessive ROS (FIGS. 3a and b) and declined membrane potential (FIG. 3c) under tBHP-exposed nucleus pulposus cells. This study is the first trial of amobarbital as an inhibitor of mitochondrial ROS production in intervertebral disc with oxidative damage. Amobarbital as an inhibitor of complex I has been introduced as having an inhibitory effect on other tissues. Ambrosio et al., showed that amobarbital significantly reduced oxygen radical concentration by blocking mitochondrial respiration in cardiac ischemia (Ambrosio et al., 1997; Chen et al., 2006). Moreover, articular chondrocytes treated with amobarbital exhibited healthy anabolism with maintained cell viability and extracellular matrix, and eventually prevented PTOA after articular joint injuries. In this study, the synergistic effects of combined NAC and amobarbital treatment on inhibiting mitochondrial damage was investigated. Although the two drugs work on the same mechanism as an inhibitor of mitochondrial electron transport complex I, they may show pharmacokinetic differences in terms of drug stability or rates of cellular uptake, and NAC has to be metabolized before it can exert its effects, whereas amobarbital does not (Bhagavan et al., 2002; Ezerina et al., 2018). There was no significant difference between NAC/amobarbital mixture and either drug alone in MitoSOX Red and JC-1 experiments (FIG. 3).

An exogenous inducer, tBHP, is known to cause lipid peroxidation and deplete glutathione, and has been used to simulate oxidative stress in the target cells. The disc cells were exposed to various concentrations and durations of treatments in previous reports. Xu et al. (2019) used 400 μM tBHP for 6 h, which showed 54.3% viability, in human nucleus pulposus cells, and Gap et al. (2019) showed notably cell apoptosis (approximately 7 times than control) in rat nucleus pulposus cells treated with 100 μM tBHP for 24 h. In contrast, 30 μM tBHP induced dramatic cell apoptosis and mitochondrial dysfunction in rat nucleus pulposus cells (6-7 times than control) (Lin et al., 2020). In this study, we used 50 μM tBHP because most rabbit nucleus pulposus cells became apoptotic and/or necrotic cells after 100 μM tBHP exposure and no superoxide production was observed at 25 μM tBHP.

An intervertebral disc organ culture system was established to evaluate acute discal injuries and therapeutic effects of amobarbital. This simple transverse cutting of the intervertebral disc generated measurable ROS production, as reflected in increased DHE staining (FIG. 5). However, in contrast to the post-tBHP treatment with amobarbital in in vitro nucleus pulposus cell culture, treatment with amobarbital significantly reduced ROS in injured nucleus pulposus tissues (FIG. 5). These results indicate that our discal injury model may be suitable for screening the therapeutic effect of amobarbital on disc degeneration. In the literature, the signaling response to oxidative stress through Nrf2 is dependent on cell type and induction method. In contrast, tBHP-induced oxidative stress was not enough to attenuate Nrf2 expression compared with control. Nevertheless, the Nrf2 expression in the cytosol of nucleus pulposus cells pre-treated with amobarbital and then treated with tBHP was close to control. Unlike tBHP exposure, physical injury to intervertebral discs led to a high proportion of nuclear localized Nrf2, indicating activation of the Nrf2 pathway by injury. Interestingly, amobarbital and NAC clearly induced dramatic positive expression of Nrf2 in the cytosol of most of nucleus pulposus cells in injured discs while preventing nuclear translocation (FIGS. 5c and d). This result implies that amobarbital can protect against oxidative stress generated by acute discal injury in a similar manner to NAC.

In order to identify the protective effect of amobarbital on cells undergoing oxidative stress, we further examined MAPK signaling pathways. Nrf2 and phosphorylation of ERK signaling pathways are involved in tBHP-exposed rat nucleus pulposus cells, but phosphorylation of JNK and p38 pathway was not detected in that study (Wang et al., 2019). Another study showed that phosphorylation of ERK and JNK as well as p38 were activated in human nucleus pulposus cells (Dimozi et al., 2015). Moreover, ERK, JNK, and p38 were highly phosphorylated in TNF-α-exposed rat AF cells, and oral administration of NAC attenuated phosphorylation of p38, but not ERK and JNK (Suzuki et al., 2015). Similar with human nucleus pulposus cells, rabbit nucleus pulposus cells treated with tBHP highly expressed all MAPK signaling pathways (ERK, JNK, and p38), while NAC or amobarbital treatment significantly suppressed MAPK expression in this study (FIG. 4).

Acute discal injury for the ex vivo study was created in only lumbar intervertebral discs and evaluated for oxidative stress in the nucleus pulposus tissues and therapeutic potential of amobarbital, because the incidence of lumbar disc degeneration is higher than that of thoracic disc degeneration due to increased mobility in the lumbar spine (McInerney et al., 2000). This approach targeting oxidative stress with amobarbital, which is locally injectable into the injured disc, can be applicable to prevent disc degeneration in human clinical practice.

### Conclusions

These findings suggest that amobarbital treatment represents a promising therapeutic option to protect against oxidative stress in nucleus pulposus cells, which has been shown to contribute to injury-induced disc degeneration. Therefore, this strategy targeting oxidative stress in nucleus pulposus, which is safer, less costly, and more effective, can be useful for disc degeneration prevention.

### Example 2

Intervertebral disc (IVD) degeneration (IDD) is clinically related to chronic low back pain (LBP), which is the most prevalent musculoskeletal disorder and results in large economic and social costs (Hong et al., 2013; Katz et al., 2006). Since the pathogenesis of IDD involves a complex signaling network and multiple effector molecules, the molecular mechanisms of IDD are largely unclear and there are no effective therapies (Urban et al., 2003; Vo et al., 2011). Thus, development of new measures for the prevention and treatment of IDD is urgent. Recent studies have clearly demonstrated that oxidative stress contributes to progression of IDD, and antioxidant therapy should be a promising therapeutic strategy for IDD (Dimozi et al., 2015; Suzuki et al., 2015).

Rabbit thoracic and lumbar spine columns were obtained from rabbit cadavers without radiographic evidence of trauma. Under sterile condition, total spine motion segments consisting of 2 vertebrae and an IVD were dissected by removing the posterior elements and soft tissues. The segments were pre-cultured in Dulbecco's modified eagle medium/nutrient mixture F-12 (DMEM/F-12) supplemented with 10% fetal bovine serum, 50 μg/ml L-ascorbate, and antibiotics in hypoxic conditions, and then exposed to 100 μM Tert-butyl hydrogen peroxide (tBHP: Sigma-Aldrich) for 30 minutes. After washing, the IVDs were treated with 10 mM NAC for 24 hours. They were then stained with dihydroethidium (DHE: Thermo Fisher Scientific) to visualize oxidant production, and Calcein AM to characterize viability via confocal microscopy. After imaging, the IVDs were processed for superoxide dismutase 2 (SOD2; Abcam) and nuclear factor-like 2 (Nrf2; Abcam) immunohistochemical (IHC) staining.

Rabbit NP cells were isolated using a collagenase-digestion method and incubated in hypoxic culture condition (5% O₂/CO₂ at 37° C.). The cells (2×104 in 96-well plate, 6 per group) were exposed to 100 μM tBHP for 30 minutes and treated with 10 mM NAC and/or 2.5 mM amobarbital for 30 minutes. A standard mitochondrial stress test was conducted to determine basal oxygen consumption rate (OCR) using an XF96 Extracellular Flux Analyzer (Seahorse Bioscience). Basal OCR was normalized to cell number. Data were analyzed by one-way ANOVA with the Tukey post-hoc test using SPSS software and expressed as average±standard deviation. Statistical significance was set at p<0.05.

Fresh rabbit IVDs were harvested and oxidative stress in the nucleus pulposus (NP) was induced by tBHP. In confocal images, there was a dramatic reduction of DHE positive cells after NAC treatment (FIG. 10B) compared with non-treated control (FIG. 10A). The effect of antioxidant(s) was confirmed in SOD2 and Nrf2 IHC staining which showed NAC treatment reduced oxidative damage in the NP cells (FIGS. 11B and D). Treatment with both amobarbital and NAC revealed a synergistic effect of suppressing basal OCR in oxidation-damaged rabbit NP cells (p=0.0443) (FIG. 12).

Although the ex vivo and in vitro studies showed therapeutic potential of antioxidants targeting NP cell metabolism after oxidative injuries, a controlled delivery system is needed to establish for in vivo local delivery of antioxidants. In conclusion, amobarbital and NAC prevent oxidative damage of IVD cells and ECM, and the combination treatment showed a synergistic effect in reducing oxidative stress in NP cells.

This study explored the extent to which an antioxidant therapeutic approach can prevent disc degeneration that is safer, less costly, and more effective than current conventional surgical approaches. Thus, the disclosure provides for a minimally-invasive local delivery procedure of controlled-released antioxidants via temperature-sensitive hydrogel is employed in vivo.

### Example 3

Harvest of rabbit IVDs: Rabbit thoracic and lumbar spine columns were obtained from rabbit cadavers without radiographic evidence of trauma. Under sterile condition, total spine motion segments consisting of 2 vertebrae and an IVD were dissected by removing the posterior elements and soft tissues. The segments were pre-cultured in Dulbecco's modified eagle medium/nutrient mixture F-12 (DMEM/F-12) supplemented with 10% fetal bovine serum, 50 μg/ml L-ascorbate, and antibiotics in hypoxic conditions.
Antioxidant effect of NAC on the IVDs: Nucleus pulposus (NP) were exposed to 100 μM Tert-butyl hydrogen peroxide (tBHP: Sigma-Aldrich) for 30 minutes. After washing, the IVDs were treated with 10 mM NAC for 24 hours. They were then stained with dihydroethidium (DHE: Thermo Fisher Scientific) to visualize oxidant production, and Calcein AM to characterize viability via confocal microscopy. After imaging, the IVDs were processed for superoxide dismutase 2 (SOD2; Abcam) and nuclear factor-like 2 (Nrf2: Abcam) immunohistochemical (IHC) staining.
Antioxidant effect of NAC and amobarbital on the NP cells: Rabbit NP cells were isolated using a collagenase-digestion method and incubated in hypoxic culture condition (5% O₂/CO₂ at 37° C.). The cells (2×10⁴ in 96-well plate, 6 per group) were exposed to 100 μM tBHP for 30 minutes and treated with 10 mM NAC and/or 2.5 mM amobarbital for 30 minutes. A standard mitochondrial stress test was conducted to determine basal oxygen consumption rate (OCR) using an XF96 Extracellular Flux Analyzer (Seahorse Bioscience). Basal OCR was normalized to cell number.
Statistical analysis: Data were analyzed by one-way ANOVA with the Tukey post-hoc test using SPSS software and expressed as average±standard deviation. Statistical significance was set at p<0.05.

### Results

Fresh rabbit IVDs were harvested and oxidative stress in the NP was induced by tBHP. In confocal images, there was a dramatic reduction of DHE positive cells after NAC treatment (FIG. 10B) compared with non-treated control (FIG. 10A). The effect of antioxidant(s) was confirmed in SOD2 and Nrf2 IHC staining which showed NAC treatment reduced oxidative damage in the NP cells (FIGS. 11B and D). Treatment with both amobarbital and NAC revealed a synergistic effect of suppressing increases in basal OCR in oxidation-damaged rabbit NP cells (p=0.0443) (FIG. 12).

### Summary

In conclusion, amobarbital and NAC prevent oxidative damage of IVD cells and ECM, and the combination treatment showed a synergistic effect in reducing oxidative stress in NP cells.

This study explores the extent to which a new antioxidant therapeutic approach can prevent disc degeneration that is safer, less costly, and more effective than current conventional surgical approaches.

### Example 4

In order to determine release behavior, 2.5 mM amobarbital was encapsulated in hydrogel mixture of F-127 (poly(ethylene oxide)-poly(propylene oxide)-poly(ethylene oxide) tri-copolymer) and hyaluronic acid. A lyophilized PF-72® (TGelBio, Seoul, South Korea) was added 10 ml, 11.765 ml, and 13.333 ml of amobarbital to make 20% wt/vol/0.5% wt/vol, 7%/0.425, and 15%/0.375% of F127/hyaluronic acid, respectively. Gel-One® (Zimmer Inc., Warsaw, Ind.) with 0.5 and 0.75% wt/vol was also prepared. Amobarbital (2.5 mM) was dissolved in phosphate-buffered saline (PBS: pH 7.4) as a control. The amobarbital/hydrogel and amobarbital (1.2 ml) were placed in a dialysis tube with 10,000 molecular weight cut-off (Float-A-Lyzer) and submerged in 12 ml PBS at 37° C. with 300 rpm shaking. The release buffer (100 μl) is collected at 0.25, 0.5, 1, 2, 4, 6, 24, 48, and 72 hours and diluted with 80 μl methanol. The amount of amobarbital at each time point was measured by high-performance liquid chromatography at 220 nm.

The group of amobarbital (no hydrogel) showed approximately 100% detection at 6 h (FIG. 13). In contrast, amobarbital in hydrogel, PF-72n and Gel-One®, showed sustained release for 72 h. Higher concentration of F127 and/or hyaluronic acid delayed the release of amobarbital.

### Example 5

Rabbit thoracic and lumbar intervertebral discs (T11/L2) were obtained from New Zealand White rabbit cadavers (approximately 10-months old) without radiographic evidence of trauma. Under sterile conditions, the intervertebral discs were dissected by removing the posterior elements and soft tissues. The discs were punctured using a 20-gauge needle with 3 mm-depth and the needle was held in place for 5 seconds. Discal injury was confirmed by herniated nucleus pulposus through the needle hole. After the puncture, 5 μl amobarbital in hydrogel (17% wt/vol F-127/0.425% wt/vol hyaluronic acid: PF-72®) or hydrogel vehicle was injected into the nucleus pulposus using a 30-gauge Hamilton syringe with a stopper (Thermo Fisher Scientific, Waltham, Mass., USA). The feasibility of our delivery approach was verified by adding Richardson's solution (blue dye) to visualize the distribution in the nucleus pulposus (FIG. 14). The blue dye was uniformly distributed in whole area of nucleus pulposus without minimal back flow through the needle.

### Example 6

### Methods

### Amobarbital-Loaded Hydrogel

In order to determine the release behavior of Amo from a hydrogel, 2.5 mM Amo (Amytal® sodium; Bausch Health, Bridgewater, N.J., USA) was encapsulated in a composite hydrogel composed of F-127 (poly(ethylene oxide)-poly(propylene oxide)-poly(ethylene oxide) tri-copolymer) and hyaluronic acid (HA). In brief, a lyophilized PF-72® (TGel Bio, Seoul, Republic of Korea) was added to 10 ml, 11.765 ml, or 13.333 ml of Amo diluted in distilled water to make 20% (w/v)/0.5% (w/v), 17%/0.425, or 15%/0.375% of F-127/HA, respectively. Separately, Amo (2.5 mM) was dissolved in phosphate-buffered saline (PBS: pH 7.4) as a control. The Amo/hydrogel and Amo (1.2 ml) solutions were placed in a dialysis tube with 10,000 molecular weight cut-off (Float-A-Lyzer®; Spectrum Chemical Manufacturing, New Brunswick, N.J., USA) and submerged in 12 ml PBS at 37° C. with 300 rpm shaking. The release buffer (100 μl) was collected at 0.25, 0.5, 1, 2, 4, 6, 24, 48, and 72 hours (h) and diluted with 80 μl methanol. The amount of amobarbital at each time point was measured by high-performance liquid chromatography incorporated with ultra-violet spectroscopy (HPLC-UV) (Agilent 1100 Series; Agilent Technologies, Santa Clara, Calif., USA) at 220 nm.

### Ex Vivo IVD Puncture Model

Overall study design is illustrated in FIG. 15A. A total of 36 rabbit thoracic and lumbar motion segments (T11/L2) including two vertebral bodies and one IVD were obtained from New Zealand White (NZW) rabbit cadavers (approximately 10-months old) without radiographic evidence of trauma by X-ray. Under sterile conditions, the segments were dissected by removing the posterior elements and soft tissues. The discs were punctured using a 20-gauge needle with 3 mm-depth, and the needle was held in place for 5 seconds. Discal injury was confirmed by herniated nucleus pulposus (NP) through the needle hole. After the puncture, 5 μl hydrogel (17% F-127/0.425% HA) with or without 2.5 mM Amo was injected into the NP using a 30-gauge Hamilton syringe with a stopper (Thermo Fisher Scientific, Waltham, Mass., USA). The feasibility of our delivery approach was validated by injecting Richardson's staining (blue color) to visualize the distribution in the NP (FIG. 15B). The spine motion segments were cultured in Dulbecco's modified eagle medium/nutrient mixture F-12 (DMEM/F-12; Thermo Fisher Scientific) supplemented with 10% fetal bovine serum (Thermo Fisher Scientific), 50 μg/ml L-ascorbate (Sigma-Aldrich, St. Louis, Mo., USA), 100 U/ml penicillin-streptomycin (Thermo Fisher Scientific), and 2.5 μg/ml amphotericin B (Sigma-Aldrich) in hypoxic culture condition (5% O₂/CO₂ at 37° C.). After 2 and 7 days, the segments were harvested for histology analysis.

### Histological Evaluation of Degenerative Disc Changes

All histological procedures were conducted in the department of Orthopedics and Rehabilitation's Histopathology Service Center (University of Iowa, Coralville, Iowa, USA). Rabbit spine motion segments were fixed in 10% buffered neutral formalin, decalcified in 5% buffered formic acid, paraffin-embedded, and coronally sectioned with a 5-μm thickness. The sections were then stained with Weigert's iron hematoxylin (Electron Microscopy Sciences, Hatfield, Pa., USA) for 6 minutes (min), 0.02% (w/v) Fast Green (Sigma-Aldrich) for 2 min, and 1% (w/v) Safranin-O (Sigma-Aldrich) for 6 min using a Gemini AutoStainer (Thermo Fisher Scientific). The stained slides were imaged using a VS220 Digital Slide Scanner (Olympus, Center Valley, Pa., USA).

In order to evaluate for IVD degenerative changes, a modified histological classification was adapted based on two well-established grading systems. Four graders independently and blindly scored twice according to 4 categories; morphology of the AF, border between the AF and NP, cellularity of the NP, and matrix of the NP (Table 4). The scales were ranging 0-8 points, and higher points indicated a severe degenerated disc. The central and lateral discs were separately assessed to compare between directly injured area (central) and adjacent area (lateral). The reliability of histology grading system was evaluated for inter-observer (between observers) and intra-observer (between two scores from one observer) correlation coefficients using Kendall's τ-b test (>0.7: excellent, 0.501-0.7: good, 0.301-0.5: moderate, ≤0.3: low). Any grader with low range of correlation(s) (less than 0.3) was excluded from further mean calculation.

### TUNEL Assay

Apoptotic cells in the NP were detected using a terminal deoxynucleotidyl transferase dUTP nick end labelling (TUNEL) assay (Abcam, Cambridge, Mass., USA) according to the manufacturer's instructions. Briefly, the sections were permeabilized by Proteinase K (1/100 in distilled water) for 20 min, and endogenous peroxidase activity was quenched by 3% (v/v) hydrogen peroxide in methanol for 5 min. Then, the sections were labeled with terminal transferase (TdT) enzyme for 1.5 h, detected with blocking buffer for 30 min, developed with working 3,3′-diaminobenzidine (DAB) solution for 15 min, and counterstained with methyl green for 1.5 min. After scanning images, the number of positive cells in each tissue was counted and normalized by the data from the 2-days intact group.

### Voltage-Dependent Anion Channel 1 (VDAC1) Immunohistochemical (IHC) Stain

VDAC1 IHC stain was performed using an automated staining instrument, Discovery ULTRA system (Roche Diagnostics, Indianapolis, Ind., USA). In brief, the sections were pre-treated with 0.24 casein U/ml Protease 3 (Roche Diagnostics) for 16 min and quenched with 3% (v/v) hydrogen peroxide in methanol for 12 min. Poly clonal rabbit anti-human VDAC1 (LSBio, Seattle, Wash., USA) was added using a 1:20 dilution for 32. The sections were incubated with OmniMap anti-rabbit horseradish peroxidase (HRP) conjugate (Roche Diagnostics) for 16 min and then developed with ChromoMap DAB (Roche Diagnostics). Lastly, the sections were counterstained with hematoxylin II and Bluing (Roche Diagnostics). The stained slides were imaged using an Olympus VS220 Digital Slide Scanner.

To quantify the amount of IHC positive expression, we followed a semi-quantification procedure using ImageJ Fiji software (Version 1.53c; NIH, Bethesda, Md., USA) [20]. Briefly, an image was deconvoluted with a H DAB (hematoxylin and DAB) vector option, and a mean grey intensity value and the number of nuclei from DAB and H images, respectively, were measured (FIG. 21). The mean grey intensity value was divided by the number of nuclei, and then normalized by the data from the 2-days intact group.

### Statistics

The scatter plots were expressed as the mean values with the standard deviation using GraphPad Prism (Version 8.1.2; San Diego, Calif., USA). Continuous normally distributed data from quantified TUNEL, VDAC1, and Amo release profile were analyzed using one-way ANOVA with the Tukey post-hoc test. Non-parametric data from histological grading system scores were analyzed using Kruskal-Wallis test on ranks with Dunn-Bonferroni post-hoc pairwise comparisons. SPSS Statistics software (Version 27; IBM, Armonk, N.Y., USA) was used to perform all parametric and non-parametric analyses, and statistical significance was set at p<0.05.

### Results

### In Vitro Amobarbital Release Profile

The Amo release profile in a hydrogel was evaluated in various concentrations of F-127 and HA. Cumulative percent release of Amo at 72 h was 76.8±10.4%, 92.0±13.1%, and 94.8±4.6% in 20%/0.5%, 17%/0.425%, and 15%/0.375% of F-127/HA hydrogel, respectively (FIG. 16). Amo was more slowly released in higher concentration of F-127 and HA, however, there was no significant differences among the groups. In contrast, Amo (No Hydrogel in FIG. 16) was completely dissolved within 24 h.

### Histological Evaluation of Degenerative Disc Changes

FIG. 17 is representative images of intact (no disc puncture), hydrogel (named HG) or Amo in hydrogel (named Amo+HG) injection after a disc puncture. Histologically, the rabbit IVDs injected with hydrogel only showed severe degenerative changes at day 7. Especially, the disc puncture at the central disc induced the loss of gelatinous NP which replaced with condensed matrix. In contrast, there was no apparent structural changes in both intact and Amo+HG.

Degenerative disc changes of gelatinous extracellular matrix (ECM) and cellular morphology were examined separately in central and lateral NP (FIGS. 18 and 19, respectively). In the central NP, where the area was injured with a disc needle puncture, NP ECM showed irregular distribution with moderate GAG loss and herniated matrix-free space in both HG and Amo+HG at 2 days (FIG. 18A: upper panel). In contrast to the morphology of notochordal cells, which were scattered in irregular clumps, in the intact NP, the cells injured with a needle puncture changed to clustered cells (blue arrowheads) (FIG. 18A: lower panel). However, the population of clustered cells was higher in HG group. At 7 days, gelatinous ECM was replaced with thick fibrous ECM (yellow arrow bars) in HG group, while there was no dramatic change in both intact and Amo+HG (FIG. 18B: upper panel). In the fibrous ECM of HG group, endplate chondrocytes surrounded by peri-cellular matrix (white asterisks) were observed along the fiber alignment (FIG. 18b: lower panel).

Four graders independently scored twice for a modified histological grading system for intervertebral disc degeneration (Table 4). Reliability of inter- and intra-observer correlation coefficients is shown in Table 5. In most comparisons, the coefficients were ranged from excellent to moderate (greater than 0.3). However, a grader (D) showed low range of correlation coefficients (less than 0.3), therefore, the data was excluded for further quantification of grading. Histological scores in the central discs were plotted in FIGS. 18c and d for 2 and 7 days, respectively. A disc puncture in HG group induced dramatic degenerative changes (5.28±1.82 at 2 days and 6.23±0.59 at 7 days), and the scores were significantly higher than those in intact group (HG vs. Intact; p<0.001 at 2 days and p=0.001 at 7 days). On the other hand, Amo+HG prohibited the progression of IDD with statistical differences (p=0.045 at 2 days and p=0.031 at 7 days vs. HG).

The progression of cellular and ECM changes in the lateral NP was similar with those in the central NP (FIG. 19). Notochordal cells were morphologically changed to clustered cells at 2 days in HG group (blue arrowheads) (FIG. 19A: lower panel), and fibrous tissues (yellow arrow bars) were observed at 7 days post-HG injection in the periphery of lateral NP (FIG. 19B: upper panel). Especially, endplate chondrocytes (white asterisks) migrated through the alignment of inner AF lamellae (FIG. 19B: lower panel). In contrast to HG, Amo treatment prohibited these degenerative disc changes at both 2 and 7 days. In grading scores, the inhibitory effect of Amo on IDD was statistically significant (p=0.018 at 2 days and p=0.006 at 7 days vs. HG) (FIGS. 19C and D). There were no significant differences between intact and Amo+HG.

### Apoptosis of NP Cells

The apoptosis of NP cells was evaluated using TUNEL assay at 2 and 6 days (FIGS. 20A and B). Compared to intact group, a disc puncture induced increased number of TUNEL positive cells in both groups of HG and Amo+HG (FIG. 20A). In particular, HG group showed statistically significant increase of apoptosis at 7 (p=0.007 vs. Intact) days in the quantified data (FIG. 20B). NP cells treated with Amo exhibited diminished number of cell apoptosis with a significant difference at 7 days (p=0.023 vs. HG).

The cell apoptosis via a disc puncture was validated by a pro-apoptotic protein, VDAC1 (FIGS. 20C and D). Positive VDAC1 expression was localized in the cytoplasm of NP cells (FIG. 20C; brown), and the intensity normalized by total cells was the highest in HG group at 2 days. Compared to intact control, VDAC1 expression was approximately 5.2 and 3.8 times higher in HG (p=0.001 vs. Intact) and Amo+HG (p=0.022 vs. Intact), respectively, at 2 days (FIG. 20D). Although there was no significant difference between HG and Amo+HG at 7 days. Amo alleviated the expression of VDAC1. The VDAC1 expression in HG was decreased, but it was significantly higher than that of the intact (p=0.005 vs. Intact).

### Discussion

The goal of the study was to evaluate the preventive effects of Amo on the progression of IDD using a disc injury model in ex vivo organ culture of rabbit spinal motion segments. Degenerative discs were created by a needle puncture, and Amo targeting oxidative stress was delivered with a temperature-sensitive F-127/HA hydrogel which showed sustained release for 3-4 days (FIG. 16). Our results revealed that Amo treatment after a discal injury prohibited morphologic changes of NP notochordal cells, structural changes of ECM, endplate chondrocyte migration, and cell apoptosis compared with hydrogel only group (FIG. 17-20).

In this study, rabbit motion segments were chosen based on Seol et al., reporting the species difference of degenerative disc changes between NZW rabbit and Sprague-Dawley (SD) rat during ex vivo organ culture of intact motion segments. In contrast to organ culture of rat IVDs, which showed the loss of notochordal cells and GAG, migration of endplate cells into the NP, and increased over-expression of matrix metalloproteinase, the integrity of rabbit IVDs was stable without any histological sign of degenerative changes up to 14 days. However, catabolic enzymes including matrix metalloproteinase-3 (MMP-3) and fibronectin were instable at day 14. Based on these data, we examine the Amo effects up to 7 days. In contrast to intact rabbit IVDs, a disc puncture into the rabbit NP induced dramatically cellular and matrix changes within 7 days in this study (FIG. 17-19). Notochordal cells were replaced to clustered cells at 2 days, and then endplate chondrocytes migrated into the NP with disorganization and loss of gelatinous NP tissue at 7 days. These degenerative changes were significantly inhibited by Amo injection.

Amo is primarily applied as a sedative-hypnotic to treat sleep disorder and as a preanesthetic agent. It has also been used to prevent post-traumatic osteoarthritis (PTOA due to its inhibitory capacity of mitochondrial electron transport complex I. Recently, Amo stability was evaluated according to United States Pharmacopeia (USP) guidelines (data not shown). The Amo was mixed with commercially available HA hydrogel (Gel-One®; Zimmer Biomet, Warsaw, Ind., USA) and showed uniform distribution in the hydrogel and stability in human biologic fluids and various conditions (temperature, light, etc.). Thus, Amo can be stably delivered in the hydrogel and applicable to use in clinics for IDD prevention in the future.

2.5 mM Amo was used based on previous results of cell viability. Rabbit NP cells showed no cytotoxicity at lower than 2.5 mM Amo for 2 h, and the viability was maintained under oxidative condition which was treated with 50 μM tBHP. When the Amo was loaded in a F-127/HA hydrogel, the drug was linearly released for 72 h with approximately 92% cumulative release (FIG. 16). In general, the drug-loaded hydrogel can be administrated using higher dosage of drug depended on the degree and duration of sustained release. However, we used same concentration rather than higher concentration because the density of notochordal cells in the NP tissue (124.2 cells/mm² in bovine NP) is relatively lower than that in in vitro cell culture condition (approximately 500 cells/mm²).

VDAC1 functions as a mitochondrial gatekeeper on the outer membrane of mitochondria. It plays important roles in (1) metabolic and energy cross-talk between the mitochondria and cytosol, (2) Ca²⁺ regulation, and (3) apoptosis-mediated release of cytochrome c. Over-expression of VDAC1 has been reported in several diseases such as cancer, neurodegenerative diseases, type 2 diabetes, and cardiac diseases. In a previous rat organ culture study, the expression of pro-apoptotic proteins including VDAC1 was increased in the degenerative discs. Moreover, VDAC1 is also involved in regulating oxidative stress. Under excessive oxidative stress, ROS are released through VDAC1 opening and activated mitogen-activated protein kinases (MAPKs) signaling molecules, especially extracellular signal-regulated kinase (ERK), c-JUN N-terminal kinase (JNK), and p38 translocated to mitochondria causing mitochondrial dysfunction and cell apoptosis. In a previous study, Amo suppressed the phosphorylation of MAPKs in in vitro NP cell culture with tBHP-induced oxidative condition. Similarly, the amount of VDAC1 expression in Amo+HG showed a decreased trend at 2 days compared with HG (FIGS. 20C and D). This result implicates that Amo inhibits VDAC1 opening and subsequent MAPKs activation, eventually reducing the apoptosis of NP cells from oxidative stress-mediated discal injuries.

The Ex vivo organ culture system of IVDs is a superior option to study IDD in terms of simulating physiological discal injury and maintaining cells and ECM in their natural context compared with in vitro cell culture system. Nevertheless, the organ culture system has several limitations such as evaluating only short-term Amo efficacy up to 7 days, slow progression of IDD due to absence of physiological mechanical loading and systemic effect of injury-related inflammation, potential instability of hydrogel in physiologic loading condition, and any side effect of Amo on other organs. Therefore, the protective effects of Amo on IDD are eventually needed to validate through an in vivo rabbit punch mod

In summary, Amo injection loaded in a temperature-sensitive hydrogel prohibited cellular and structural disc changes in NP cells during ex vivo organ culture of rabbit spine motion segments with a disc puncture. Therefore, Amo treatment targeting oxidative stress may prevent, inhibit or treat degenerative disc degeneration.

TABLE 4
A modified histological grading system for intervertebral disc degeneration.

| | II. Border between the anulus |
|---|---|
| I. Morphology of the Anulus fibrosus | fibrosus and nucleus pulposus |
| 0. Normal, pattern of fibrocartilage lamellae | 0. Normal |
| without ruptured fibers and without a | |
| serpentine appearance anywhere within | |
| the anulus | |
| 1. Ruptured or serpentined patterned fibers | 1. Minimally interrupted |
| in less than 30% of the annulus | |
| 2. Ruptured or serpentined patterned fibers | 2. Moderate/severe interruption |
| in more than 30% of the annulus | |
| | IV. Matrix of the nucleus |
| III. Cellularity of the nucleus pulposus | pulposus |
| 0. Normal cellularity: mainly notochordal | 0. Normal gelatinous appearance |
| cells | |
| 1. Slightly decreased cellularity: mixture of | 1. Slight condensation of the |
| notochordal cells and cell clusters | extracellular matrix |
| 2. Moderately/severely decreased (>50%) | 2. Moderate/severe condensation |
| cellularity: mainly cell clusters | of the extracellular matrix |

TABLE 5
Reliability of histology grading system with Kendall’s τ-b correlation
coefficients (>0.7: excellent, 0.501-0.7: good, 0.301-0.5: moderate, ≤0.3: low).

| Central disc at 2 days | | | | | Central disc at 7 days | | | | |
|---|---|---|---|---|---|---|---|---|---|
| Observer | A | B | C | D | Observer | A | B | C | D* |
| A | 0.553 | 0.808 | 0.682 | 0.744 | A | 0.759 | 0.540 | 0.807 | 0.296* |
| B | | 0.902 | 0.549 | 0.656 | B | | 0.922 | 0.517 | 0.279* |
| C | | | 0.768 | 0.717 | C | | | 0.793 | 0.542 |
| D | | | | 0.732 | D | | | | 0.624 |

| Lateral disc at 2 days | | | | | Lateral disc at 7 days | | | | |
|---|---|---|---|---|---|---|---|---|---|
| Observer | A | B | C | D | Observer | A | B | C | D |
| A | 0.724 | 0.592 | 0.454 | 0.685 | A | 0.634 | 0.747 | 0.508 | 0.871 |
| B | | 0.777 | 0.438 | 0.535 | B | | 0.924 | 0.529 | 0.621 |
| C | | | 0.790 | 0.606 | C | | | 0.682 | 0.445 |
| D | | | | 0.904 | D | | | | 0.745 |

*The grader with low range of coefficients (less than 0.3) was excluded for further analysis.

### REFERENCES

Adams et al., Spine, 25:1625 (2000).
Aldakkak et al., Cardiovasc. Res., 77:406 (2008).
Ambrosio et al., J. Biol. Chem., 268:18532 (1993).
Bartynski et al., Interv. Neuroradiol., 19:110 (2013).
Battie et al., Spine, 20:2601 (1995).
Bhagavan N V. CHAPTER 14—Electron Transport and Oxidative Phosphorylation. In: Bhagavan N V, editor. Medical Biochemistry (Fourth Edition). San Diego: Academic Press; 2002. p. 247-74.
Cai et al., Osteoarthritis Cartilage, 23:1763 (2015).
Camara et al., Front Physiol., 8:460 (2017).
Carter et al., Chiropr. Man. Therap., 21:13 (2013).
Chen et al., Int. J. Biochem. Cell. Biol., 59:1 (2015).
Chen et al., J. Pharmacol. Exp. Ther., 316:200 (2006).
Chevallier et al., Hepatology, 20:349 (1994).
Coleman et al, Sci Transl. Med., 10, (2018).
Crowe and Yue, Bio. Protoc., 9, (2019).
Dimozi et al., Eur. Cell. Mater., 30:89 (2015).
Dudli et al., Spine J., 14:1256 (2014).
Elfawy and Das, Life Sci., (2018).
Ezerina et al., Cell. Chem. Biol., 25:447 (2018).
Fang and Maldonado, Adv. Cancer Res., 138:41 (2018).
Feng et al., Oxid. Med. Cell. Longev., 2017:5601593 (2017).
Gao et al., Am. J. Transl. Res., 11:5956 (2019).
Hirst et al., Biochem. Soc. Trans., 36:976 (2008).
Hong et al., Spine (Phila Pa. 1976), 38:75 (2013).
Itoh et al., Genes Dev., 13:76 (1999).
Jiang et al., Sci. Rep., 4:7456 (2014).
Kattoor et al., Curr. Atheroscler. Rep., 19:42 (2017).
Katz, J. Bone Joint Surg. Am., 88 Suppl 2:21 (2006).
Kim et al., Biomed. Res. Int., 2015:812949 (2015).
Kim et al., Int. J. Radiat. Oncol. Biol. Phys., 88:1142 (2014).
Kobayashi et al., Mol. Cell. Biol., 24:7130 (2004).
Krupkova et al., Oxid. Med. Cell. Longev., 2016:7031397 (2016).
Krupkova et al., Eur. Cell. Mater., 28:372 (2014).
Lee et al., J. Orthop. Res., 33:1743 (2015).
Lee et al., J. Biomed. Mater. Res. A., 92:378 (2010).
Lei et al., Am. J. Transl. Res., 9:900 (2017).
Lepetsos and Papavassiliou, Biochim. Biophys. Acta., 1862:576 (2016).
Li et al., J. Transl. Med., 4:77 (2016).
Li et al., Spine (Phila Pa. 1976), 33:2586 (2008).
Li et al., PeerJ., 4:e1992 (2016).
Lin et al., Free Radic. Biol. Med., 150:109 (2020).
Ma et al., Osteoarthritis Cartilage, 21:2030 (2013).
Mahmoud et al., Oxid. Med. Cell. Longev., 2017:2508909 (2017).
Maroudas et al., J. Anat., 120:113 (1975).
Masuda et al., Spine (Phila Pa. 1976), 30:5 (2005).
McInerney and Ball, Neurosurg. Focus. 9:el (2000).
Miller et al., Spine (Phila Pa. 1976), 13:173 (1988).
Nguyen et al., J. Biol. Chem., 284:13291 (2009).
Oh et al., nt. J. Pharm., (2019).
Rutges et al., Osteoarthritis Cartilage. 21:2039 (2013).
Sahoo et al, Clin. Sci. (Lond), 130:317 (2016).
Sambrook et al., Arthritis. Rheum., 42:366 (1999).
Schroeder et al., Evid. Based Spine Care J., 3:33 (2012).
Sena et al., Front Physiol., 9:1668 (2018).
Seol et al., J. Orthop. Res., 31:838 (2013).
Seol et al., Spine J. 21:1021 (2021).
Seol et al., J. Biomed. Mater. Res. B. Appl. Biomater., 101:1508 (2013).
Seol et al., J. Orthop. Res., 36:2439 (2018).
Son et al., J. Signal Transduct., 2011:792639 (2011).
Stewart et al., Transl. Res., 153:224 (2009).
Suzuki et al., Arthritis Res. Ther., 17:316 (2015).
Urban and Roberts, Arthritis Res. Ther., 5:120 (2003).
Victor et al., Curr. Pharm. Des., 17:3947 (2011).
Vo et al., J. Orthop. Res., 34:1289 (2016).
Wang et al., Age (Dordr), 35:1741 (2013).
Wang et al., Cell. Prolif., 4:381 (2014).
Wang et al., Aging (Albany N.Y.), 11:7961 (2019).
Weiler et al., Eur. Spine J., 21 Suppl 6:S810 (2012).
Wu et al., Exp. Mol. Med., 51:1 (2019).
Xu et al., Exp. Mol. Med., 51:1 (2019).
Yamamoto et al., Mol. Cell. Biol., 28:2758 (2008).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to prevent, inhibit or treat intervertebral disc disease in a mammal, comprising locally administering to a disc of the mammal an effective amount of a hydrogel composition comprising hyaluronic acid and an amount of amobarbital or a derivative thereof effective to prevent, inhibit or treat intervertebral disc degeneration, and optionally a poloxamer.

2. The method of claim 1 wherein the composition is injected.

3. The method of claim 1 wherein the hyaluronic acid is about or greater than 0.5 MDa.

4. The method of claim 1 wherein the hyaluronic acid is present in the composition from about 0.01% (wt/vol) and up to about 2.0% (wt/vol).

5. The method of claim 1 wherein the hyaluronic acid is present at about 0.2% (wt/vol) and up to about 1.0% (wt/vol).

6. The method of claim 1 wherein the composition includes the poloxamer from about 15% (wt/vol) and up to about 20% (wt/vol).

7. The method of claim 1 wherein the hydrogel comprises amobarbital, pentobarbital, secobarbital, phenobarbital, adenosine diphosphate ribose, or metformin, or a derivative thereof.

8. The method of claim 1 wherein disc degeneration is inhibited.

9. The method of claim 8 wherein the disc is a thoracic disc, a lumbar disc or a cervical disc.

10. The method of claim 1 wherein the administration reduces reactive oxygen species (ROS) production in the nucleus pulposus.

11. The method of claim 1 wherein the administration is within 4 days of spinal injury or surgery.

12. The method of claim 1 wherein the mammal is a human.

13. The method of claim 1 wherein the composition is a thermoresponsive or temperature sensitive hydrogel.

14. The method of claim 1 wherein the mammal has an injury in the nucleus pulposus, annulus fibrosus, or endplate.

15. The method of claim 1 wherein the mammal has disc herniation.

16. The method of claim 1 wherein a syringe is employed to administer the composition.

17. The method of claim 16 wherein the syringe has a 22 to 24-gauge needle.

18. A method to prevent, inhibit or treat spinal degeneration in a mammal, comprising locally administering to a spine of the mammal an effective amount of a hydrogel composition comprising hyaluronic acid, hydroxypropylcellulose, karaya gum (KG), guar gum (GUG), or gellan gum (GEG) and a compound in an amount that reversibly inhibits respiratory enzyme complex, and optionally a synthetic polymer.

19. The method of claim 18 wherein the composition comprises amobarbital, pentobarbital, secobarbital, phenobarbital, barbital, adenosine diphosphate ribose, or metformin, or a derivative thereof.

20. The method of claim 18 wherein the composition comprises hyaluronic acid of about or greater than 0.5 M Dalton or from about 0.01% (wt/vol) and up to about 2.0% (wt/vol) in the composition.
